(19) United States
(12) Patent Application Publication
(10) Pub. No.: US 2013/0098834 A1
(43) Pub. Date: Apr. 25, 2013

(54) MEDIUM, DEVICES AND METHODS

(71) Applicant: Jonas Axelsson, Stockholm (SE)

(72) Inventor: Jonas Axelsson, Stockholm (SE)

(73) Assignee: JJK MEDICAL LTD., Jersey Channel Islands (GB)

(21) Appl. No.: 13/805,810

(22) PCT Filed: Jun. 17, 2011
(86) PCT No.: PCT/EP2011/060130

§ 371 Date: Dec. 21, 2012

Related U.S. Application Data
(60) Provisional application No. 61/357,231, filed on Jun. 22, 2010.

(30) Foreign Application Priority Data
Jun. 22, 2010 (EP) 10166799.6

(51) Int. Cl. C07K 14/705

(52) U.S. Cl. CPC C07K 14/705
USPC 210/502.1; 530/395; 530/350

(57) ABSTRACT

A separation medium is provided, comprising at least one megalin polypeptide and/or at least one cubilin polypeptide immobilized on a support. Also provided are devices comprising the separation medium, as well as methods and uses employing the separation medium for extracorporeal removal of low molecular weight proteins, or fragments or derivatives thereof, from complex biological fluids.

### FIELD OF THE INVENTION

The present invention relates to a separation medium comprising polypeptides immobilized on a support. The invention also relates to a medical device comprising such a separation medium and a dialysis device comprising such a medical device, as well as uses of such devices, for example for hemodialysis, hemofiltration and/or hemodiafiltration. Further, the invention relates to a method for extracorporeal removal of a low molecular weight protein from a complex biological fluid, and a method of treatment through removal of low molecular weight proteins, or fragments or derivatives thereof, from the blood of a patient.

### BACKGROUND

The kidney is the most important organ for the clearance of unwanted polypeptides in the blood of mammals (Brenner B M (2003) Brenner & Rector's The Kidney (7^{th} edition)), and loss of kidney function results in a marked accumulation of polypeptides (Naseeb U et al (2008) Blood Purif. 26(6): 561-8). Kidney dysfunction is dichotomized into acute renal failure (characterized by a deterioration of renal function over a period of hours or days, and resulting in the failure of the kidney to excrete nitrogenous waste products and to maintain fluid and electrolyte homeostasis) and chronic kidney disease (CKD) (signifying a permanent loss of these functions to a lesser or greater degree) (Brenner B M (2003), supra). Acute renal failure occurs mainly due to a reduced blood supply to the kidney filtration apparatus and is most common in patients with hypovolemic shock (Brenner B M (2003), supra), while CKD has multiple causes and has now reached epidemic proportions with 10-12% of the Western population showing signs of CKD (Wen C P et al (2008) Lancet 371(9631):2173-82). A range of underlying diseases cause CKD, the quantitatively most important being diabetic nephropathy due to diabetes mellitus, nephrosclerosis due to hypertension and glomerulonephritis (Brenner B M (2003), supra). Despite disparate beginnings, the later-stage CKD kidney dysfunction phenotype is remarkably similar between etiologies, and with the advent of dramatically reduced glomerular filtration rate sooner or later requires regular dialysis or renal transplantation for survival. Despite the advances in renal replacement therapy, both patients with acute renal injury or chronic kidney disease suffer from an increase in circulating polypeptides (Naseeb U et al (2008), supra) thought to derive from reduced tubular clearance (Moestrup S K et al (1995), J Clin Invest. 96(3):1404-13; Vinge L et al (2010), Nephrol Dial Transplant, advance e-publication doi: 10.1093/ndt/gfq044), and to be related to quality of life and survival. This symptom is not addressed by current therapies.

A large number of polypeptides of small or intermediate molecular weight, as well as some of large molecular weight, are filtered through the renal glomerulus and end up in the renal tubules. There, they are bound by scavenger receptors on the luminal surface of tubular cells and taken up by endocytosis (Moestrup S K et al (1995), supra). These polypeptides are then returned to the circulation through transcytosis or broken down in the peritubular cells for recycling into amino acids for fresh protein synthesis (Christensen E I et al (2002) Nat Rev Mol Cell Biol. 3(4):256-66; Russo L M et al (2007) Kidney Int., 71(6):505-13). Thus, these peritubular receptors are essential for the physiological protection from urinary loss of a plethora of proteins and polypeptides essential for normal body function.

The multiligand, endocytic receptors megalin and cubilin are colocalized in the renal tubule. Both receptors are important for normal tubular reabsorption of proteins filtered in the glomerulus, including in albuminuria (Russo L M et al (2007), supra; Vinge L et al (2010), supra).

Current treatment options for kidney dysfunction concentrate on the homeostasis of small molecules, primarily water and salts, through specific binders (eg. potassium and phosphate binders) or their non-specific removal through dialysis (eg. hemodialysis and peritoneal dialysis). Peritoneal dialysis utilizes the peritoneal membrane as a filter. Hemodialysis, as well as hemofiltration and hemodiafiltration, utilizes an extracorporeal circuit and a synthetic, usually plastic, filter. All current methods of treating non-specific loss of kidney function are based on molecular size filters to remove unwanted and potentially hazardous molecules. Additionally, a number of more specific methods exist to remove targeted compounds in specific situations. These include protein A columns designed to remove leukocytes before transplantation (Weiss L et al (1986) Appl Biochem Biotechnol. 13(2):87-96), columns coated with antibodies against immunoglobulin light chains to remove such light chains in myeloma patients (Hutchinson C A et al (2007) J Am Soc Nephrol. 18(3):886-95), and semi-specific heparin-coated devices for removing heparin-binding cytokines in inflamed states (Axelsson J et al (2010) ASAIO J. 56(1):48-51).

US patent application publication 2004/0235161 focuses on the use of an intracorporeal artificial kidney, which comprises a sponge sheet with cells having megalin expressed on the surface. Whole renal tubular cells are utilized for the cleansing of blood.

PCT application publication WO2003/102593 focuses on the use of megalin for the protection from exogenously administered polypeptides.

There is a need for blood purification techniques which result in the specific removal of toxic, physiological, and pathological polypeptides from complex biological fluids, e.g. blood, during dialysis treatment.

### DISCLOSURE OF THE INVENTION

It is an object of the present disclosure to alleviate at least some of the problems associated with the prior art techniques.

In particular, it is an object of the disclosure to provide a separation medium which allows removal of low molecular weight proteins and/or fragments or derivatives thereof from a fluid.

It is also an object of the disclosure to provide a medical device which allows selective removal of low molecular weight proteins and/or fragments or derivatives thereof from a complex biological fluid.

It is another object of the disclosure to provide a medical device which allows restoration of the composition of a complex biological fluid.

Yet another object of the disclosure is to provide a method for extracorporeal removal of a low molecular weight protein and/or fragment or derivative thereof from a complex biological fluid.

The above mentioned objects, as well as other objects that will be apparent to a person skilled in the art when presented with the present disclosure, are each addressed by at least one of the different aspects of the present invention.

In a first aspect thereof, the present invention provides a separation medium comprising a) at least one megalin polypeptide, and/or b) at least one cubilin polypeptide, immobilized on a support.

In the context of the present invention, the term “separation medium” refers to a medium for separation, for example a column or a filter.

Throughout the present disclosure, the term “megalin polypeptide” refers to a megalin receptor or a variant, domain, fragment or derivative thereof retaining at least one function of the megalin receptor. For example, the at least one function can be a binding function for at least one ligand. The megalin receptor is a member of a family of receptors with structural similarities to the low density lipoprotein receptor (LDLR), and is also known as “low density lipoprotein-related protein 2” (LRP2) (Christensen E I et al (2002), supra; Cui S et al (2010) Am J. Physiol. Renal Physiol. 298(2):335-345). The megalin receptor is a multiligand binding receptor found in the plasma membrane of many absorptive epithelial cells. The protein functions to mediate endocytosis of ligands leading to degradation in lysosomes or transcytosis. In humans, the protein is encoded by the LRP2 gene. A non-limiting example of the amino acid sequence of the human megalin receptor is disclosed in the appended sequence listing as SEQ ID NO:1. As exemplified in the experimental section below, different fragments of the megalin receptor retain at least one function of the megalin receptor, and may be useful in the different aspects of the present invention, by themselves or in any combination of such fragments with each other, with the full-length receptor or with other fragments. Examples of such fragments or domains are denoted herein as MEG1 (SEQ ID NO:2), MEG2 (SEQ ID NO:3), MEG3 (SEQ ID NO:4), MEG4 (SEQ ID NO:5), MEG5 (SEQ ID NO:6), MEG6 (SEQ ID NO:7), MEG7 (SEQ ID NO:8), MEG8 (SEQ ID NO:9), MEG9 (SEQ ID NO:10), MEG10 (SEQ ID NO:11) and MEG5-8 (SEQ ID NO:12). A “megalin polypeptide” may, however, also designate a similar protein, fragment, domain or derivative, which fulfils at least one of functions of the megalin receptor. The amino acid sequence of such a polypeptide may for example be related to an amino acid sequence specifically disclosed herein by one or more conservative substitution mutations, in which an amino acid residue in the disclosed sequence has been replaced by another amino acid residue in the same group of amino acid residues sharing physico-chemical properties. Such groupings are well known to the person of skill in the art of protein engineering. Put another way, a “megalin polypeptide” may resemble a specific disclosed megalin polypeptide sequence by a degree of similarity or identity of at least 80%, such as at least 85%, such as at least 90% or such as at least 95%.

In a similar fashion, in the context of the present invention, the term “cubilin polypeptide” refers to a cubilin receptor or a fragment thereof retaining at least one function of the cubilin receptor. For example, the at least one function can be a binding function for at least one ligand. In vivo, cubilin (also known as cubulin, intestinal intrinsic factor receptor, intrinsic factor-vitamin B12 receptor and 460 kDa receptor) is located within the epithelium of intestine and kidney (Christensen E I et al (2002), supra; Kozyraki R et al (1998) Blood 91 (10): 3593-3600; U.S. Pat. No. 6,586,389). In humans, the protein is encoded by the CUBN gene. A non-limiting example of the amino acid sequence of the human cubilin receptor is disclosed as SEQ ID NO:13 in the sequence listing. As exemplified in the experimental section below, different fragments of the cubilin receptor retain at least one function of the cubilin receptor, and may be useful in the different aspects of the present invention, by themselves or in any combination of such fragments with each other, with the full-length receptor or with other fragments. Examples of such fragments or domains are denoted herein as CUBEGF (SEQ ID NO:14), CUB1-7 (SEQ ID NO:15), CUB5-8 (SEQ ID NO:16), CUBE-12 (SEQ ID NO:17), CUB11-17 (SEQ ID NO:18), CUB16-22 (SEQ ID NO:19) and CUB21-27 (SEQ ID NO:20). A “cubilin polypeptide” may, however, also designate a similar protein, fragment, domain or derivative, which fulfils at least one of the functions of the cubilin receptor. The amino acid sequence of such a polypeptide may for example be related to an amino acid sequence specifically disclosed herein by one or more conservative substitution mutations, in which an amino acid residue in the disclosed sequence has been replaced by another amino acid residue in the same group of amino acid residues sharing physico-chemical properties. Such groupings are well known to the person of skill in the art of protein engineering. Put another way, a “cubilin polypeptide” may resemble a specific disclosed cubilin polypeptide sequence by a degree of similarity or identity of at least 80%, such as at least 85%, such as at least 90% or such as at least 95%.

In a separation medium as disclosed in the present application, one or more megalin polypeptides, which may be the same or different, and/or one or more cubilin polypeptides, which may be the same or different, can be used.

In the context of the present invention, the term “support” refers to a surface on which at least one megalin polypeptide and/or at least one cubilin polypeptide are immobilized. For example, the support may be composed of beads or a membrane. If present, beads may be used in a column and a membrane may be used in a filter. The column or filter having megalin and/or cubilin polypeptides immobilized thereto may be used for separation of proteins, such as for example low molecular weight proteins and/or fragments or derivatives thereof.

Also in the context of the present invention, and as readily understood by the skilled person, the term “immobilized on a support” means that a species has been purposefully immobilized to the support, separately from other species that are also immobilized to the same support. In embodiments of the present invention where both megalin and cubilin polypeptides are present, the fact that these polypeptides are “immobilized on a support” means the immobilization onto the support of each polypeptide species separately. The immobilization may be indirect, such as using well-known affinity systems. Examples include the interaction between a His-tag in the respective polypeptide and a support provided with a chelating moiety such as Ni-NTA groups (or vice versa), or between a biotin group in the respective polypeptide and a support provided with streptavidin groups (or vice versa). The immobilization may also be direct, i.e. the polypeptides being covalently attached to the support. Any combination of these and other methods and means for immobilization of each of the polypeptides in question is contemplated, and may be put into practice by the skilled person without undue burden.

An advantage with a separation medium comprising at least one megalin polypeptide and/or at least one cubilin polypeptide is that the medium allows binding of proteins which are able to bind to megalin, to cubilin or to both, as the case may be, and/or of fragments or derivatives thereof. By utilizing the interaction with megalin and/or cubilin, these proteins may be at least partly removed from a fluid. For example, such a separation medium may be used in a medical device or in a dialysis device for example aimed for dialysis of a patient's blood. In this application, the amount of polypeptides of low molecular weight, and/or their fragments or derivatives, may be reduced in the blood and the original composition of low molecular weight molecules in the blood may be restored. Blood with a restored composition of proteins resembles blood which has passed a kidney and has a composition of proteins which the kidneys normally preserve in the blood.

In some embodiments of the separation medium, in which the medium comprises both megalin and cubilin polypeptides, the molar ratio between the megalin and cubilin polypeptides may be in the range of from 1:100 to 100:1, such as from 1:50 to 50:1, such as from 1:10 to 10:1, such as from 1:5 to 5:1, such as from 1:2 to 2:1 or such as 1:1. In the context of the present disclosure, the molar ratio is the ratio of the molar concentration of megalin polypeptide to the molar concentration of cubilin polypeptide. The inventor has found that the particular molar ratio of 10:1 between megalin polypeptide and cubilin polypeptide shows good results, in that many low molecular weight proteins are captured by the separation medium and removed from a complex biological fluid. However, other molar ratios also work satisfactorily. Given the teaching herein and using the above ratios as guidelines, a person of skill in the art will then be able to perform the experiments necessary to optimize the molar ratio of megalin and cubilin polypeptides in these embodiments of the separation medium according to the invention.

For the avoidance of doubt, aspects of the invention also provide a separation medium comprising immobilized megalin polypeptide only (i.e. does not comprise cubilin polypeptides) or immobilized cubilin polypeptide only (i.e. does not comprise megalin polypeptides), which in some cases may have a satisfactory effect in capturing low molecular weight proteins, and/or fragments or derivatives thereof. For example, as described in Example 11 below, a separation medium comprising immobilized megalin polypeptides (MEG5-8) binds insulin from a complex biological fluid. Furthermore, as described in Examples 12 and 13 below, a separation medium comprising immobilized full-length megalin can be used to successfully treat the blood of partially nephrectomised rats. In these examples, the megalin polypeptides are enough to obtain a separation medium useful for removal of at least one low molecular weight protein. In other situations, however, a combination of at least one megalin polypeptide with at least one cubilin polypeptide is necessary to achieve a satisfactory result.

In some embodiments, the surface density of immobilized megalin may be 1-100 000 megalin polypeptide molecules per μm², such as 100-50 000 molecules per μm², such as 1 000-20 000 molecules per μm² or such as 3 000-10 000 molecules per μm².

In some embodiments, the surface density of immobilized cubilin may be 1-100 000 cubilin polypeptide molecules per μm², such as 100-50 000 molecules per μm², such as 1 000-20 000 molecules per μm² or such as 3 000-10 000 molecules per μm².

The megalin polypeptide can be produced recombinantly or via chemical synthesis. Likewise, the cubilin polypeptide can be produced recombinantly or via chemical synthesis.

In some embodiments, the material of the support may be selected from the group consisting of glass, cellulose, cellulose acetate, chitin, chitosan, cross-linked dextran, cross-linked agarose, agar gel support, polypropylene, polyethylene, polysulfone, polyacrylonitrile, polytetrafluoroethylene, polystyrene, polyurethane, silicone and amylase coated particles. For example, the polystyrene may be selected from anilo sulfonic polystyrene and triethanolamine methyl polystyrene. In one example, the support consists of cross-linked dextran, for example Sephadex®. Other examples of supports are Sepharose® and Dynabeads®. A person skilled in the art understands that the support can be selected by trial and error given the guidelines provided herein. A support which is cheap and easy to manufacture and handle is advantageous, as well as one that keeps leakage of substances from the support material to a minimum. Furthermore, a support can be sterilized.

The support can have various forms. For example, the support may comprise beads or particles, such as microparticles or nanoparticles. In other examples, the support may comprise one or more hollow fibers. The support may be a column, for example a porous column. Furthermore, the support may be a filter.

The at least one megalin polypeptide and/or at least one cubilin polypeptide may be covalently attached to the support. The covalent attachment can be selected from the group consisting of covalent polymer grafting, plasma treatment, physisorption, chemisorption and chemical derivatization. In other examples, the at least one polypeptide may be attached to the support with CnBr coupling. In yet other examples, biotin-avidin or glutathione S-transferase (GST) coupling can be used.

In another aspect thereof, the present invention provides a medical device for extracorporeal treatment of a complex biological fluid, which comprises a separation medium as described above.

In the context of this and other aspects of the present invention, the term “complex biological fluid” refers to a water-based fluid comprising for example diverse solutes, suspended naturally occurring or manufactured polypeptides and cells. For example, a complex biological fluid may comprise proteins, salts and other molecules, for example cells. In some examples, the complex biological fluid is blood, such as for example mammalian blood, such as for example human blood. In other examples, the complex biological fluid is plasma, serum or urine. In the context of the present disclosure, plasma is the yellow liquid component of blood, in which the blood cells in whole blood would normally be suspended. It is the intravascular fluid part of extracellular fluid. It is mostly water and comprises dissolved proteins, glucose, clotting factors, mineral ions, hormones and carbon dioxide. Plasma may be prepared by spinning a tube of fresh blood containing an anti-coagulant in a centrifuge until the blood cells fall to the bottom of the tube. The plasma is then poured or drawn off. In the context of the present disclosure, serum is plasma without fibrinogen or other clotting factors (i.e. whole blood minus both the cells and the clotting factors). Serum may include all proteins not used in blood clotting and all the electrolytes, antibodies, antigens, hormones, and any exogenous substances (e.g., drugs and microorganisms).

In the context of the present invention, extracorporeal treatment refers to treatment outside the body, for example a human body. For example, extracorporeal treatment may comprise dialysis of blood. Treatment of a complex biological fluid, such as blood, can comprise removal of molecules, such as proteins, from the blood.

In some embodiments, the complex biological fluid comprises at least one low molecular weight protein, and/or a fragment or derivative thereof. The low molecular weight protein may be a part of a larger protein. In some examples, the low molecular weight protein has a molecular weight of 50 kDa or lower. In other examples, the low molecular weight protein has a molecular weight of 35 kDa or lower. In yet other examples, the low molecular weight protein has a molecular weight of 20 kDa or lower. For example, the complex biological fluid may comprise a mixture of low molecular weight proteins, and/or fragments or derivatives thereof. Furthermore, a complex biological fluid may comprise proteins larger than for example 50 kDa and other molecules than proteins.

In some embodiments of the present invention, the low molecular weight protein can be modified. Examples of such modification are glycosylation, e.g. mannose-6-phosphatation and sialylation, and leucin-rich region modification. Other examples involve the action of metalloproteinases or endoproteinases. In some examples, the low molecular weight protein or fragment or derivative thereof can be degraded.

The low molecular weight protein, or fragment or derivative thereof, can have a megalin binding motif, meaning that the protein has the ability to bind to a megalin polypeptide as defined herein. In other examples, the low molecular weight protein, or fragment or derivative thereof, can have a cubilin binding motif, i.e. have the ability to bind to a cubilin polypeptide as defined herein. The low molecular weight protein, or fragment or derivative thereof, may have at least one megalin binding motif, at least one cubilin binding motif, or a combination thereof.

In some embodiments, the low molecular weight protein can be selected from the group consisting of peptide hormones, enzymes and vitamin-binding proteins. For example, the low molecular weight protein may be selected from the group consisting of cytokines, insulin, albumin, apolipoproteins, β₂- and α₁-microglobulin, myoglobulin and immunoglobulin light chains, and fragments and derivatives thereof. In one example, the apolipoprotein may be apolipoprotein H.

In some embodiments, the medical device according to the invention may additionally comprise a size filter. Filtration is a mechanical or physical operation which is used for the separation of solids from fluids by interposing a medium through which only the fluid can pass. Mesh, bag and paper filters may be used to remove large particulates suspended in fluids while membrane processes, including microfiltration, ultrafiltration, nanofiltration, reverse osmosis and dialysis, employ synthetic membranes and may be used to separate micrometer-sized or smaller species. The size filter that may be used in a medical device according to the invention may have a cut-off of 50 kDa. In other examples, the cut-off is 35 kDa. In yet other examples, the cut-off is 20 kDa. For example, the size filter can remove large proteins, such as albumin, from a complex biological fluid. In other examples, the size filter can remove blood cells from blood.

A size filter used in some embodiments of a medical device according to the present invention may have various forms. For example, the size filter can be a fiber, a perforated sheet or a mesh type filter. The size filter can be made of a natural material. For example, the natural material can be cellulose or a derivative thereof, chitosan, carbon or aluminium oxide. In other examples, the size filter can be made from a man-made material for example selected from the group consisting of nylon 6-6, polyvinylidene fluoride, polypropylene, polytetrafluoroethylene, polyethersulfone, glass and metal. A person skilled in the art understands that the size filter can be selected by trial and error given the guidelines provided herein. The size filter is preferably cheap, sterilizable, easy to manufacture and handle, and leakage from the material of the size filter is preferably low.

In some embodiments, the medical device may further comprise a charge filter. In some examples, the charge filter only allows passage for species having an isoelectric point, pI, of ≦8. In other examples, the charge filter only allows passage for species having pI≦7. In yet other examples, the charge filter only allows passage for species having pI≦5.8.

A charge filter may have various forms. For example, the charge filter can be a fiber, a perforated sheet or a mesh type filter. The charge filter can be made of a natural material. For example, the natural material can be cellulose or a derivative thereof, chitosan, carbon or aluminium oxide. In other examples, the charge filter can be made from a man-made material for example selected from the group consisting of nylon 6-6, polyvinylidene fluoride, polypropylene, polytetrafluoroethylene, polyethersulfone, glass and metal. A person skilled in the art understands that the charge filter can be selected by trial and error given the guidelines provided herein. The charge filter is preferably cheap, sterilizable and easy to manufacture and handle, and leakage from the material of the charge filter is preferably low.

In some embodiments, the size filter and the charge filter is the same filter. In some examples, the size filter and the charge filter are two different filters. For example, the size filter may be placed before the charge filter in a medical device. In this example, the complex biological fluid added to the medical device reaches the size filter before the charge filter. In other examples, the charge filter is placed before the size filter in a medical device.

In some embodiments of the present invention, the medical device may be sterilized before use. Sterilization refers to any process that effectively kills or eliminates transmissible agents (such as fungi, bacteria, viruses, spore forms, etc.) from a surface. Sterilization may be performed with heat, chemicals, irradiation, high pressure or filtration. A widely-used method for heat sterilization is the autoclave. Autoclaves commonly use steam heated to 121-134° C. In other examples, the device can be sterilized with gamma radiation. Gamma rays are very penetrating and are commonly used for sterilization of disposable medical equipment, such as syringes. Other alternatives include using sterilizing solutions such as ethanol. In some embodiments of the present invention, the device may not be sterilized as a whole, but instead assembled in a sterile environment using previously sterilized parts.

In another aspect thereof, the present invention provides a dialysis device for extracorporeal treatment of a complex biological fluid, which comprises a medical device as described herein. The dialysis device may comprise other parts than the medical device as described herein. For example, a dialysis device according to the present invention may comprise more than one medical device. More than one medical device may be arranged in parallel or in series.

In another aspect thereof, the present invention provides a method for extracorporeal removal of a low molecular weight protein from a complex biological fluid, comprising the steps:

a) providing a sample of complex biological fluid containing a low molecular weight protein having a binding affinity for megalin and/or cubilin,
b) bringing said sample into contact with a separation medium, medical device or dialysis device as disclosed above, under conditions allowing binding of said low molecular weight protein to said at least one megalin polypeptide and/or at least one cubilin polypeptide,
c) separating said sample from said support, such that at least part of the total amount of said low molecular weight protein initially present in said sample is retained on the support, and
d) recovering said sample containing a reduced amount of said low molecular weight protein.

The method for extracorporeal removal of at least one low molecular weight protein from a complex biological fluid may be used as a method for extracorporeal treatment of a complex biological fluid. In the method for extracorporeal treatment a separation medium according to the invention as described herein, may be used, for example comprised in a medical device according to the invention as described herein.

In some embodiments of the method of the present invention, the complex biological fluid may be blood. In some examples, the blood is mammalian blood, for example human blood. In other examples, the complex biological may be plasma, serum or urine.

The sample of complex biological fluid can be obtained by using for example a blood dialysis circuit. The blood dialysis circuit may be connected to a patient suffering from a kidney disease.

The sample is brought into contact with the at least one megalin polypeptide and/or at least one cubilin polypeptide. For example, the sample may be brought into contact with a separation medium comprising either of or both megalin and cubilin polypeptides, or a medical device or dialysis device comprising such a separation medium.

During the separation, at least one low molecular weight protein, or fragment or derivative thereof, is contemplated to bind to the megalin polypeptide and/or cubilin polypeptide and be retained from the complex biological fluid. The fluid flows through the separation medium while the at least one low molecular weight protein, or fragment or derivative thereof, binds to the immobilized polypeptides on the support in the separation medium. During separation, at least part of the amount of low molecular weight proteins, or fragments or derivatives thereof, in the complex biological fluid can be retained and subsequently removed from the complex biological fluid.

The complex biological fluid can be recovered after passage through the separation medium. The recovered complex biological fluid will have a changed composition (and amount) of proteins compared to the composition (and amount) of proteins of the complex biologic fluid entering the separation medium.

One advantage with the method for extracorporeal removal of low molecular weight proteins, or fragments or derivatives thereof, from a complex biological fluid is that the method resembles the function of a normally functioning kidney. A person with a malfunctioning kidney is likely to have a problem with increased amounts of low molecular weight proteins, or fragments or derivatives thereof, in the blood, causing severe problems such as amyloidosis (increased concentration of β-2 microglobulin in the blood) or endoplasmic reticulum stress (increased concentration of megalin and/or cubilin binding residues). By using the method of extracorporeal removal of low molecular weight proteins, or fragments or derivatives thereof, from a complex biological fluid, such as blood, the amount of proteins and the composition of proteins of the fluid can be restored to a state resembling the content of the blood of a person with normally functioning kidneys.

Furthermore, the method for extracorporeal removal of low molecular weight proteins from a complex biological fluid can be used to prevent renal failure by reducing the amount of low molecular weight proteins or fragments or derivatives thereof in the blood. For example, the method for extracorporeal removal of low molecular weight proteins from blood can be used to reduce an increased concentration of myoglobulins in the blood which can be caused by muscle trauma. The increased concentration of myoglobulins in the blood may cause renal failure. In another example, the method for extracorporeal removal of low molecular weight proteins from blood can be used to reduce the amount of circulating immunoglobulin light chains associated with blood malignancies such as myeloma.

For the avoidance of doubt, aspects of the invention also provide a method for extracorporeal removal of low molecular weight proteins, or fragments or derivatives thereof, from a complex biological fluid using a separation medium comprising immobilized megalin polypeptide only (i.e. not cubilin) or immobilized cubilin polypeptide only (i.e. not megalin), which in some cases may have a satisfactory effect in capturing low molecular weight proteins. In other situations, however, a combination of at least one megalin polypeptide with at least one cubilin polypeptide is necessary to achieve a satisfactory result.

In some embodiments, the low molecular weight protein, or fragment or derivative thereof, may have a molecular weight of 50 kDa or lower. In other examples, the low molecular weight protein, or fragment or derivative thereof, has a molecular weight of 35 kDa or lower. In yet other examples, the low molecular weight protein, or fragment or derivative thereof, has a molecular weight of 20 kDa or lower.

In some embodiments of the present invention, the low molecular weight protein, or fragment or derivative thereof, can be modified. Without being bound by any specific scientific theory, some examples of such modification are glycosylation, mannose-6-phosphatation, leucin-rich region modification and sialylation. Other examples involve metalloproteinases or endoproteinases. In some examples, the low molecular weight protein, or fragment or derivative thereof, can be degraded.

In embodiments of the method according to the invention, the low molecular weight protein, or fragment or derivative thereof, may have at least one megalin binding motif, at least one cubilin binding motif, or a combination thereof.

In some embodiments, the low molecular weight protein can be selected from the group consisting of peptide hormones, enzymes and vitamin-binding proteins. For example, the low molecular weight protein may be selected from the group consisting of cytokines, insulin, albumin, apolipoproteins, β₂- and α₁-microglobulin, and immunoglobulin light chains. In one example, the apolipoprotein may be apolipoprotein H.

In some embodiments, the method further comprises a step of subjecting the sample to a size filtration step, wherein high molecular weight components can be removed from the sample before performing step b). In the context of the present invention, the term “components” refers to proteins or other molecules present in a complex biological fluid. For example, the high molecular weight components can have a molecular weight of 50 kDa or higher. In other examples, the high molecular weight components have a molecular weight of 35 kDa or higher. In yet other examples, the high molecular weight components have a molecular weight of 20 kDa or higher.

In some embodiments, the method further comprises subjecting the sample to a charge filtration step, wherein components having a pI of no more than 8 are removed from the sample before performing step b). In other examples, the removed components may have a pI of no more than 7.0. In yet other examples, the removed components may have a pI of no more than 5.8.

In some embodiments, the size filtration and the charge filtration are performed simultaneously. The size filter and the charge filter may be the same. In other examples, the size filtration may be performed before the charge filtration. In yet other examples, the charge filtration is performed before the size filtration.

In some embodiments, the method may further comprise a step e) wherein the retained low molecular weight protein is eluted. The eluted proteins can be collected and analyzed. For example, the amount of proteins and the type of proteins may be of interest for determining the disease status of a patient or for determining the treatment of a patient with a kidney disease. The medical device or the dialysis device may be reused more than once. The device may be subjected to elution between two additions of complex biological fluid.

The present invention also provides a method for treatment of a mammalian subject suffering from a condition caused or aggravated by a low molecular weight protein, or fragment or derivative thereof, comprising the steps:

a) extracting blood from the subject,
b) removing low molecular weight protein, or fragment or derivative thereof, from said extracted blood using a method of extracorporeal removal as described above, such that at least part of the total amount of said low molecular weight protein, or fragment or derivative thereof, initially present in said blood is retained on the support, and
c) reintroducing the blood, containing a reduced amount of said low molecular weight protein, or fragment or derivative thereof, into the bloodstream of the subject.

The subject suffering from a condition caused or aggravated by a low molecular weight protein, or fragment or derivative thereof, may be a patient suffering from a kidney disease. A patient suffering from a kidney disease may have one or two malfunctioning kidney(s). A possible outcome of malfunctioning kidneys may be an increased amount of low molecular weight proteins, or fragments or derivatives thereof, in the blood.

For example, the extraction and the reintroduction of blood may be performed in a continuous loop. The loop may comprise a part of the bloodstream of the subject. In other examples, the extraction of blood may be performed using a dialysis device with an external blood circuit.

In other examples, the method for treatment may be performed inside a human body where the blood is in contact with a device comprising megalin and/or cubilin polypeptides. The device may be arranged inside the human body. Blood exiting the device may have a reduced amount of proteins compared to when entering the device.

In another aspect thereof, the present invention provides a medical or dialysis device which can be used for hemodialysis, hemofiltration and/or hemodiafiltration. The device may be used in series and/or in parallel with a blood circuit. In some examples, the blood circuit may be a dialysis apparatus.

The device may be used for extracorporeal removal of a low molecular weight protein, or fragment or derivative thereof, from a complex biological fluid. The complex biological fluid may be blood, plasma, serum or urine as described herein. The complex biological fluid may comprise at least one low molecular weight protein, or fragment or derivative thereof, as described herein.

The low molecular weight protein, or fragment or derivative thereof, may be a peptide hormone, enzyme or a vitamin-binding protein. For example, the low molecular weight protein may be an inflammatory cytokine. In another example, the low molecular weight protein may be an immunoglobulin light chain. For example, myeloma causes light chain deposition in the kidneys and usage of a device according to the present invention might be advantageous in order to decrease the amount of immunoglobulin light chains in the blood.

In some examples, the device may be used for restoration of the composition of a complex biological fluid. A person suffering from a kidney disease may have increased amount of modified proteins, or fragments or derivatives thereof, in the blood. A large part of the modified proteins, or fragments or derivatives thereof, is not removed (at least not to a satisfactory extent) from the blood by the kidneys because of the kidneys' malfunction. A device according to the present disclosure may be used in order to restore the composition of the blood.

The device according to the present invention can be used for a method of treatment or prevention of a subject at risk of suffering from renal failure. For example, the device can be used in order to reduce the amount of aggregating low molecular weight proteins, or fragments or derivatives thereof, in the blood of a subject showing symptoms of renal failure.

In one embodiment of the present invention, a device according to the present invention can be used for obtaining functional proteins from a bioreactor. After isolation of proteins and separation by for example using an affinity column, a device according to the present invention can be used for separating dysfunctional proteins from functional proteins. The device according to the present invention can be used as an extra step in obtaining isolated proteins after production in a bioreactor.

In other examples, a device according to the present invention may be used in therapy for sepsis. For example, the device may be used for reducing the amount of cytokines in the blood of a subject showing symptoms of sepsis.

In yet other examples, a device according to the present invention can be used to treat acute renal failure. Acute renal failure can be caused by accumulation of myoglobulin following e.g. muscle trauma. The device according to the present invention can be used for reducing the amount of myoglobulin of a subject showing symptoms of acute renal failure.

A medical or dialysis device according to the present disclosure may be used in parallel or in series with another medical or dialysis device connected to an extracorporeal blood circuit. For example, the other medical or dialysis device may be a dialysis blood filter or an extracorporeal blood oxygenation device.

### Itemized Listing of Embodiments

The following is a non-limiting and itemized listing of embodiments of the present disclosure, presented for the purpose of describing various features and combinations provided by the invention in certain of its aspects.

### Items:

1. A separation medium comprising at least one megalin polypeptide immobilized on a support.
2. A separation medium according to item 1, wherein said megalin polypeptide is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12, and amino acid sequences having an identity of at least 80%, such as at least 85%, such as at least 90%, such as at least 95%, thereto.
3. A separation medium according to item 2, wherein said megalin polypeptide is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12.
4. A separation medium according to any one of the preceding items, wherein the surface density of immobilized megalin is 1-100 000 megalin polypeptide molecules per μm².
5. A separation medium according to item 4, wherein said surface density of immobilized megalin is 3 000-10 000 megalin polypeptide molecules per μm².
6. A separation medium according to any one of the preceding items, wherein the at least one megalin polypeptide is produced recombinantly or via chemical synthesis.
7. A separation medium comprising at least one cubilin polypeptide immobilized on a support.
8. A separation medium according to item 7, wherein said cubilin polypeptide is selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, and amino acid sequences having an identity of at least 80%, such as at least 85%, such as at least 90%, such as at least 95%, thereto.
9. A separation medium according to item 8, wherein said cubilin polypeptide is selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20.
10. A separation medium according to any one of the preceding items, comprising at least one megalin polypeptide and at least one cubilin polypeptide immobilized on a support.
11. A separation medium according to item 10, wherein the molar ratio between said megalin polypeptide and said cubilin polypeptide is in the range of from 1:100 to 100:1.
12. A separation medium according to item 11, wherein the molar ratio between said megalin polypeptide and said cubilin polypeptide is in the range of from 1:50 to 50:1, such as in the range of from 1:10 to 10:1.
13. A separation medium according to item 12, wherein the molar ratio between said megalin polypeptide and said cubilin polypeptide is 10:1.
14. A separation medium according to any one items 7-13, wherein the surface density of immobilized cubilin is 1-100 000 cubilin polypeptide molecules per μm².
15. A separation medium according to item 14, wherein said surface density of immobilized cubilin is 3 000-10 000 cubilin polypeptide molecules per μm².
16. A separation medium according to any one of items 7-15, wherein the at least one cubilin polypeptide is produced recombinantly or via chemical synthesis.
17. A separation medium according to any one of the preceding items, wherein the material of said support is selected from the group consisting of glass, cellulose, cellulose acetate, chitin, chitosan, cross-linked dextran, cross-linked agarose, agar gel support, polypropylene, polyethylene, polysulfone, polyacrylonitrile, polytetrafluoroethylene, polystyrene, polyurethane, silicone and amylose coated particles.
18. A separation medium according to item 17, wherein said material is polystyrene, and said polystyrene is selected from anilosulfonic polystyrene and triethanolamine methyl polystyrene.
19. A separation medium according to item 17, wherein said material is cross-linked dextran.
20. A separation medium according to any one of items 17-19, wherein said support comprises beads.
21. A separation medium according to item 20, wherein said beads are microparticles.
22. A separation medium according to item 20, wherein said beads are nanoparticles.
23. A separation medium according to any one of the preceding items, wherein said at least one megalin polypeptide and/or said at least one cubilin polypeptide and/or both are covalently attached to said support.
24. A separation medium according to item 23, wherein said covalent attachment is selected from the group consisting of covalent polymer grafting, plasma treatment, physisorption, chemisorption and chemical derivatization.
25. A medical device for extracorporeal treatment of a complex biological fluid, comprising a separation medium according to any one of the preceding items.
26. A medical device according to item 25, wherein said complex biological fluid is blood.
27. A medical device according to items 25-26, wherein said complex biological fluid comprises a low molecular weight protein, or a fragment or derivative thereof.
28. A medical device according to item 27, wherein said low molecular weight means having a molecular weight of 50 kDa or lower.
29. A medical device according to item 28, wherein said low molecular weight protein means having a molecular weight of 35 kDa or lower.
30. A medical device according to any one of items 27-29, wherein said low molecular weight protein is modified.
31. A medical device according to any one of items 27-30, wherein said low molecular weight protein has a megalin binding motif.
32. A medical device according to any one of items 27-31, wherein said low molecular weight protein has a cubilin binding motif.
33. A medical device according to any one of items 27-32, wherein said low molecular weight protein is selected from the group consisting of peptide hormones, enzymes, immunoglobulin light chains, myoglobulin and vitamin-binding proteins, and fragments and derivatives thereof.
34. A medical device according to item 33, wherein said low molecular weight protein is selected from the group consisting of cytokines, insulin, albumin, apolipoproteins, β₂- and α₁-microglobulin, immunoglobulin light chains, myoglobulin, and oxygen binding proteins, and fragments and derivatives thereof.
35. A medical device according to any one of items 25-34, wherein said device is sterilized before use.
36. A medical device according to any one of items 25-35, wherein said device comprises a size filter.
37. A medical device according to item 36, wherein said size filter has a cut-off of 50 kDa.
38. A medical device according to item 37, wherein said cut-off is 35 kDa.
39. A medical device according to any one of items 36-38, wherein said size filter is a fiber, a perforated sheet or a mesh type filter.
40. A medical device according to any one of items 36-39, wherein said size filter is made from a natural material, for example selected from the group consisting of cellulose or a derivative thereof, chitosan, carbon or aluminium oxide.
41. A medical device according to any one of items 36-39, wherein said size filter is made from a man-made material, for example selected from the group consisting of nylon 6-6, polyvinylidene fluoride, polypropylene, polytetrafluoroethylene, polyethersulfone, glass and metal.
42. A medical device according to any one of items 25-41, wherein said device comprises a charge filter.
43. A medical device according to item 42, wherein said charge filter only allows passage for species having pI≦8.
44. A medical device according to item 43, wherein said charge filter only allows passage for species having pI≦5.8.
45. A medical device according to any one of items 42-44, wherein said charge filter is made from a natural material, for example selected from the group consisting of cellulose or a derivative thereof, chitosan, carbon and aluminium oxide.
46. A medical device according to any one of items 42-44, wherein said charge filter is made from a man-made material, for example selected from the group consisting of nylon 6-6, polyvinylidene fluoride, polypropylene, polytetrafluoroethylene, polyethersulfone, glass and metal.
47. A medical device according to any one of items 36-46, wherein said size filter and said charge filter, when both present, are the same filter.
48. A dialysis device for extracorporeal treatment of a complex biological fluid, comprising a medical device according to any one of items 25-47.
49. Method for extracorporeal removal of a low molecular weight protein, or fragment or derivative thereof, from a complex biological fluid, comprising the steps:
   a) providing a sample of complex biological fluid containing a low molecular weight protein, or fragment or derivative thereof, having a binding affinity for megalin and/or cubilin,
   b) bringing said sample into contact with a separation medium according to any one of items 1-24 or a device according to any one of items 25-48, under conditions allowing binding of said low molecular weight protein, or fragment or derivative thereof, to said at least one megalin polypeptide and/or said at least one cubilin polypeptide,
   c) separating said sample from said support, such that at least part of the total amount of said low molecular weight protein, or fragment or derivative thereof, initially present in said sample is retained on the support, and
   d) recovering said sample containing a reduced amount of said low molecular weight protein, or fragment or derivative thereof.
50. Method according to item 49, wherein said complex biological fluid is blood.
51. Method according to item 50, wherein said blood is mammalian blood.
52. Method according to item 51, wherein said mammalian blood is human blood.
53. Method according to item 49, wherein said complex biological fluid is plasma.
54. Method according to item 49, wherein said complex biological fluid is urine.
55. Method according to any one of items 49-54, wherein said low molecular weight protein, or fragment or derivative thereof, has a molecular weight of 50 kDa or lower.
56. Method according to item 55, wherein said low molecular weight protein, or fragment or derivative thereof, has a molecular weight of 35 kDa or lower.
57. Method according to any one of items 49-56, wherein said low molecular weight protein, or fragment or derivative thereof, is modified.
58. Method according to any one of items 49-57, wherein said low molecular weight protein, or fragment or derivative thereof, has a megalin binding motif.
59. Method according to any one of items 49-58, wherein said low molecular weight protein, or fragment or derivative thereof, has a cubilin binding motif.
60. Method according to any one of items 49-59, wherein said low molecular weight protein is selected from the group consisting of peptide hormones, enzymes, immunoglobulin light chains, myoglobulin and vitamin-binding proteins, and fragments and derivatives thereof.
61. Method according to item 60, wherein said low molecular weight protein is selected from the group consisting of cytokines, insulin, albumin, apolipoproteins, β₂- and α₁-microglobulin, immunoglobulin light chains, myoglobulin, and oxygen binding proteins, and fragments and derivatives thereof.
62. Method according to any one of items 49-61, further comprising subjecting the sample to a size filtration step, whereby high molecular weight components are removed from the sample before performing step b).
63. Method according to item 62, wherein said high molecular weight components have a molecular weight of 50 kDa or higher.
64. Method according to item 63, wherein said high molecular weight components have a molecular weight of 35 kDa or higher.
65. Method according to any one of items 49-64, further comprising subjecting the sample to a charge filtration step, whereby components having a pI of no more than 8 are removed from the sample before performing step b).
66. Method according to item 65, wherein said removed components are components having a pI of no more than 5.8.
67. Method according to any one of items 62-66, wherein said size filtration and said charge filtration, when both present, are performed simultaneously.
68. Method according to any one of items 49-67, wherein said method further comprises a step e), wherein said retained low molecular weight protein, or fragment or derivative thereof, is eluted.
69. Method for treatment of a mammalian subject suffering from a condition caused or aggravated by a low molecular weight protein, or fragment or derivative thereof, comprising the steps:
   a) extracting blood from the subject,
   b) removing low molecular weight protein, or fragment or derivative thereof, from said extracted blood using a method according to any one of items 49-68, such that at least part of the total amount of said low molecular weight protein, or fragment or derivative thereof, initially present in said blood is retained on the support, and
   c) reintroducing the blood, containing a reduced amount of said low molecular weight protein, or fragment or derivative thereof, into the subject.
70. Method according to item 69, wherein the extraction and reintroduction of blood is performed in a continuous loop, which loop comprises a part of the bloodstream of the subject.
71. Use of a separation medium according to any one of items 1-24 for hemodialysis, hemofiltration and/or hemodiafiltration.
72. Use of a medical or dialysis device according to any one of items 25-48 for hemodialysis, hemofiltration and/or hemodiafiltration.
73. Use according to item 72, wherein said device is used in series and/or in parallel with a blood circuit.
74. Use according to item 73, wherein said blood circuit is a dialysis apparatus.
75. Use of a medical or dialysis device according to any one of items 25-48 for extracorporeal removal of a low molecular weight protein from a complex biological fluid.
76. Use of a medical or dialysis device according to any one of items 25-48 for restoration of the composition of a complex biological fluid.
77. Use according to item 76, wherein the complex biological fluid is blood.
78. Use according to item 77, wherein said blood comprises at least one low molecular weight protein, or a fragment or derivative thereof.
79. Method according to any one of items 69-70 or use according to item 78, wherein said low molecular weight protein, or fragment or derivative thereof, is modified.
80. Method according to any one of items 69-70 or use according to any one of items 78-79, wherein said low molecular weight protein is an inflammatory cytokine.
81. Method according to any one of items 69-70 or use according to any one of items 78-79, wherein said low molecular weight protein is an immunoglobulin light chain.
82. Use of a medical or dialysis device according to any one of items 25-48 in parallel and/or in series with another medical or dialysis device connected to an extracorporeal blood circuit.
83. Use according to item 82, wherein said other medical or dialysis device is a dialysis blood filter or an extracorporeal blood oxygenation device.

The use of at least one megalin polypeptide and/or at least one cubilin polypeptide for binding of low molecular weight proteins according to the invention will now be described in a non-limiting manner by the following Figures and Examples.

### BRIEF DESCRIPTION OF THE APPENDED FIGURES

FIG. 1 is a photograph of a gel showing the result of a northern blot of mRNA from renal biopsies from three different individuals. The legends are, from left to right, “Ladder”, “Biopsy 1”, “Biopsy 2” and “Biopsy 3”.

FIG. 2 is a photograph of a gel showing the result of a western blot of two polypeptides, namely MEG1 (SEQ ID NO:2), and CUB5-8 (SEQ ID NO:16). The sizes of MEG1 and CUB5-8 are both approximately 40 kDa.

FIG. 3 is a photograph of immunoblot gels of filtered (<30 kDa) blood from a healthy volunteer (Healthy) and from a patient with chronic kidney disease treated by maintenance hemodialysis (Uremic), before and after passage through a column with Sepharose® having megalin and cubilin polypeptides immobilized thereto (HEP) or through a column with Sepharose® only (CTR).

FIG. 4 shows chromatograms from reverse phase HPLC showing the results from four different samples with (“SIZE+”) and without (“SIZE−”) passage through a size exclusion column and before (“COL−”) and after (“COL+”) passage through a column with beads having immobilized thereto the polypeptides MEG3 (SEQ ID NO:4) and CUB1-7 (SEQ ID NO:15).

FIG. 5 is a diagram of the total protein content (TPC) (g/l) in samples before and after passage though a column with immobilized megalin and cubilin polypeptides with different ratios of megalin and cubilin.

FIG. 6A shows a diagram of the result from an ELISA assay, showing the change in insulin amount (pg/ml) after passage through a column with immobilized megalin polypeptide MEG5-8 (SEQ ID NO:12). The result of three different columns is shown; a control column (no antibodies added), a column with added anti-insulin antibodies and a column with added anti-megalin antibodies. FIG. 6B shows a photograph of a gel of a western blot. The first lane shows passage of complex biological fluid to which no antibodies have been added. The second lane shows passage of complex biological fluid to which anti-insulin antibodies have been added. The third lane shows passage of complex biological fluid to which anti-megalin antibodies have been added. The fourth lane shows passage of complex biological fluid to which both anti-insulin and anti-megalin antibodies have been added. For each passage, a value of TPC is presented.

FIG. 7 are pictures of 2-D gels showing the different effect of (A) MEG and (B) CTRL columns on pooled rat <30 kDa plasma proteomes.

FIG. 8 is a diagram showing frequency of typical behavior during 3 days of recordings of nephrectomised (MEG and CTRL) and non-nephrectomised (SHAM) rats, after passage of pooled blood samples through columns with (MEG) and without (CTRL and SHAM) bound megalin. For SHAM, n=4; for MEG, n=3; and for CTRL, n=3.

### EXAMPLES

In the following non-limiting Examples 1-10, the principle of using megalin and cubilin polypeptides for removing at least one low molecular weight protein from a complex biological fluid is shown. Furthermore, non-limiting Examples 11-13 show that, in certain circumstances, megalin polypeptides are enough to obtain a satisfactorily result when removing at least one low molecular weight protein from a complex biological fluid.

### Example 1

### Creation of Megalin and Cubilin cDNA

The procedure described in Andersen C B et al, (2010), Nature 464:445-448, was followed. In brief, total RNA was extracted from human renal cortex (renal biopsy from 3 individuals) using AllPrep® DNA/RNA/Protein Mini Kit (Qiagen) as instructed by the manufacturer. mRNA was isolated using the Oligotex® kit (Qiagen).

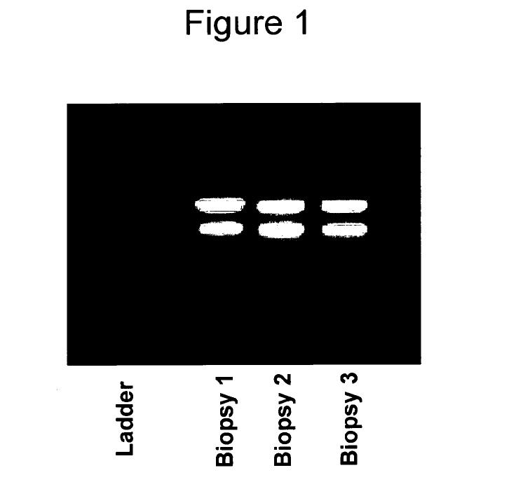

RACE was carried out to obtain cDNA using the Qiagen Reverse Transcription Kit with the primers given in Tables 1 and 2 to generate DNA encoding the indicated megalin and cubilin polypeptides (the full amino acid sequences of which are provided in the appended sequence listing), along with cleavage sites for the indicated restriction enzymes. Northern blotting was performed for quality assurance on 1′)/0 formaldehyde gels at 100 V and with 1 μg of mRNA for 1 hour and visualized with horseradish peroxidase-labeled riboprobes to check quality (FIG. 1).

TABLE 1
Primers used in RACE to obtain megalin and cubilin cDNA for
expression in E. coli cells

| Primer name | Primer sequences and restriction sites |
|---|---|
| MEGALIN_forward | GTCGACTCatggatcgcgggccggcagcag (Sal I) |
| MEGALIN_reverse | GCGGCCGCctatacttcagagtcttctttaac (Not I) |
| MEG1_forward | GAATTCtgtgacagtgcgcattttcg (EcoR I) |
| MEG1_reverse | CTCGAGccgatagcttccatccaaatttac (Xho I) |
| MEG2_forward | GTCGACTCgttctcaatgtttctgttgaaacc (Sal I) |
| MEG2_reverse | GCGGCCGCtcgatctgttccatccacg (Not I) |
| MEG3_forward | GGATCCattgtgaacagcagtctgg (BamH I) |
| MEG3_reverse | CTCGAGtcgatggtggccctccaaatcag (Xho I) |
| MEG4_forward | GTCGACTCcgacacacggtgtatgatg (Sal I) |
| MEG4_reverse | GCGGCCGCtacttcagagtcttctttaac (Not I) |
| MEG5_forward | GGATCCtgtgacagtgcgcattttc (BamH I) |
| MEG5_reverse | CTCGAGttcatccgcgtcatctgaacag (Xho I) |
| MEG6_forward | GGATCCtgctcaagtcatcagataac (BamH I) |
| MEG6_reverse | CTCGAGgcaagcatgttcgtcactg (Xho I) |
| MEG7_forward | GTCGACTCtgcggtggttaccagttcac (Sal I) |
| MEG7_reverse | GCGGCCGCtctgtctaaaccatcaaagg (Not I) |
| MEG8_forward | GAATTCcagtgtggcttattttcctt (EcoR I) |
| MEG8_reverse | CTCGAGgcggaagtttcctcccaatgtg (Xho I) |
| MEG9_forward | GGATCCattgtgaacagcagtctgg (BamH I) |
| MEG9_reverse | CTCGAGatcaacacaagtccgcttgtc (Xho I) |
| MEG10_forward | GTCGACTCgatattgatgaatgcacagag (Sal I) |
| MEG10_reverse | GCGGCCGCtacttcagagtcttctttaac (Not I) |
| MEG5-8_forward | GGATCCtgtgacagtgcgcattttc (BamH I) |
| MEG5-8_reverse | CTCGAGgcggaagtttcctcccaatgtg (Xho I) |
| CUBILIN_forward | GTCGACTCatgatgaacatgtctttaccttttc (Sal I) |
| CUBILIN_reverse | GCGGCCGCttagctgtcccaagttaatcgg (Not I) |
| CUBEGF_forward | GGATCCaaaaaggtttgcagcagcaatc (BamHI) |
| CUBEGF_reverse | CTCGAGaggaacctgacagagagctccag (XhoI) |
| CUB1-7_forward | GGATCCtgtggagagtccctctcaggaa (BamHI) |
| CUB1-7_reverse | GCGGCCGCtgtctgccggtattcagccttg (Not I) |
| CUB5-8_forward | GGATCCtgtggagaaattcttacagaac (BamHI) |
| CUB5-8_reverse | CTCGAGaccgtaaacaaaccactgct (XhoI) |
| CUBE-12_forward | GGATCCtgtttgcaagactacacagatg (BamHI) |
| CUB6-12_reverse | CTCGAGgccaaatatcttcataaatgtg (Xho I) |
| CU B11-17_forward | GGATCCtgcggaggccacatcctcacc (BamHI) |
| CU B11-17_reverse | CTCGAGctcttccatactggattcaaatcg (Xho I) |
| CUB16-22_forward | CGGCCGtgtgggggcaacgtctacatccat (Eag I) |
| CUB16-22_reverse | CTGCAGggagattatcctataggaaaact (Pst I) |
| CUB21-27_forward | GGATCCtgtggtggaatatttcattctg (BamHI) |
| CUB21-27_reverse | CTCGAGgctgtcccaagttaatcggaatgc (Xho I) |

TABLE 2
Primers used in RACE to obtain megalin and cubilin cDNA for
expression with His₁₀ tags in HEK293 cells

| Primer name | Primer sequences and restriction sites |
|---|---|
| HIS-MEGALIN_forward | CTCGAGTatggatcgcgggccggcagcag (Xho I) |
| HIS-MEGALIN_reverse | GGGCCCctatacttcagagtcttctttaac (Apa I) |
| HIS-CUBILIN_forward | GGATCCaaaaaggtttgcagcagcaatc (BamHI) |
| HIS-CUBILIN_reverse | CTCGAGgctgtcccaagttaatcggaatgc (Xho I) |

### Example 2

### Expression of GST-Coupled Megalin and Cubilin Polypeptides in E. Coli cDNA products created as described in Example 1 were ligated into pGEX-4T-3 vectors (GE Healthcare) and transformed using electroporation into Escherichia coli strain DH5a (New England Biolabs Inc.). Recycling of cDNA products was done by DNA Gel Extraction Kit (Promega). Transformed cells were identified by plating on LB agar plates containing 100 mg/ml ampicillin and incubation overnight at 37° C. A single colony was inoculated into 3 ml LB-ampicillin medium and allowed to grow overnight at 37° C. in an orbital shaker with constant shaking at 250 rpm. Next, the culture was diluted 1:100 into 200 ml fresh LB-ampicillin (100 mg/ml) medium and incubated at 37° C. with constant shaking at 250 rpm until OD₆₀₀ was approximately 0.5 (2-3 h). Protein expression was then induced by addition of IPTG to the culture to a final concentration of 0.5 mM and the culture incubated again overnight at 15° C. with constant shaking at 250 rpm. The following day, the culture was centrifuged at 6 000 g for 10 min at 4° C. to form cell pellets. Still at 4° C., the pellet was resuspended in 10 ml cold lysis buffer, sonicated on ice (at 30% amplitude with five 10 s bursts and a 30 s cooling interval between each burst) and protein release monitored by the Bradford reaction (10 μl fraction+90 μl water+1 ml Bradford reagent (Pierce); absorbance at 590 nm and 450 nm was measured, and the ratio between these used to calculate protein concentration). The lysate was cleared by centrifugation at 20 000 g for 30 min at 4° C., after which the supernatant was aliquoted and stored on ice. For quality control, the recombinant plasmids were extracted by IllustraPlasmid Prep MiniSpin (GE Healthcare) according to the manufacturer's instructions, cut using the restriction enzymes given in Table 1 for the indicated polypeptides and evaluated on the gel provided in the kit.

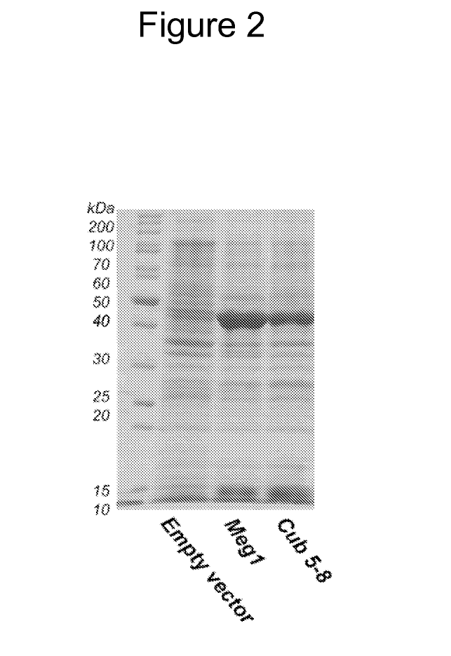

Purification of expressed constructs was done by glutathione S-transferase (GST) affinity chromatography as described in Hodneland et al, (2002), Proc Natl Acad Sci USA, 91(21):9725-9. Briefly, 2 ml of glutathione-Sepharose (Sigma) resin was mixed with cell lysate and 1 ml of 0.25 M NaCl in a glass column and stirred gently for 1 hour. Washing was done using 3 column volumes, 15 ml at each time, of a wash buffer (20 mM Tris, pH 7.5+0.25 M NaCl+2 mM EDTA+2 mM EGTA+0.03% Brij-35 (Sigma)). Protein elution was then performed using a premixed wash buffer to which had been added 20 mM glutathione (Sigma) and NaOH to make the pH 8.0. After each elution, protein yield was checked using the Bradford reaction. Pure protein (typical yields: for MEG1 9-12 mg/l culture and for CUB5-8 18-21 mg/l culture) was next eluted in PBS, concentrated using a VivaSpin 20 (Sartorius Stedim Biotech) ultrafiltration spin column at 4° C. and 3 000 g, and finally stored at −80° C. A representative sample of the western blot gels of the obtained products is shown in FIG. 2.

### Example 3

### Expression of Biotin-Coupled Megalin and Cubilin Polypeptides in E. Coli

Cysteine-biotin was synthesized as previously described (Liu et al, (2008), Mol. Biotechnol. 39(2):141-53). Briefly, 2.6 mmol N-t-Boc-S-trityl-L-cysteine, 3.1 mmol tetramethyl-O-(benzotriazol-1-yl)uronium tetrafluoroborate (TBTU) and 3.9 mmol 1-hydroxybenzotriazole were added to 50 ml of dry dimethylformamide (DMF). The mixture was stirred for 20 min at room temperature. Next, 7.8 mmol N-methyl morpholine and 2.6 mmol biotinylethylenediamine (Promega) were added. The reaction was allowed to continue for 3 h under gentle stirring, after which the solvents were evaporated in vacuum using a rotary evaporator equipped with an oil pump. This crude reaction mixture was dissolved in 200 ml of dichloromethane (DCM) and the organic layer extracted with water (3×100 ml), then dried by addition of anhydrous MgSO₄ (25 g) for 5 min with periodic stirring. The resulting solution (180 ml) was carefully decanted into a clean flask and concentrated in vacuum using the evaporator. Purification of cysteine-biotin was done by flash chromatography (4-8% v/v MeOH in DCM) over silica gel (200 g) packed in a glass column.

cDNA products created as described in Example 1 (one primer pair per batch) were ligated into pMD-18-TX vectors (Promega) and transformed using electroporation into Escherichia coli strain ER2566. Transformed cells were identified by plating on LB agar plates containing 100 mg/ml ampicillin incubated overnight at 37° C. A single colony was inoculated into 3 ml LB-ampicillin media and allowed to grow overnight at 37° C. in an orbital shaker with constant shaking at 250 rpm. Next, the culture was diluted 1:100 into 200 ml fresh LB-ampicillin (100 mg/ml) media, and incubated at 37° C. with constant shaking at 250 rpm until OD₆₀₀ was approx. 0.5 (2-3 h). Protein expression was then induced by isopropyl β-D-1-thiogalactopyranoside (IPTG) added to the culture to a final concentration of 0.5 mM and the culture incubated again overnight at 15° C. with constant shaking at 250 rpm. The following day, the culture was centrifuged at 6 000 g for 10 min at 4° C. to form cell pellets. Still at 4° C., the pellet was resuspended in 10 ml cold lysis buffer, sonicated on ice (at 30% amplitude with five 10 s bursts and a 30 s cooling interval between each burst) and protein release monitored by Bradford protein assay. The lysate was cleared by centrifugation at 20 000 g for 30 min at 4° C., after which the supernatant was aliquoted and stored on ice. Proteins were isolated by HPLC chromatography with 20 ml of chitin beads per liter culture in a column using a flow rate 0.5-1 ml/min and biotinylated on the column using 30 mM of 2-mercaptoethane sulfonic acid (Sigma) and 1 mM of cysteine-biotin. Finally, the biotinylated protein was eluted with 10 ml of PBS and stored at −80° C.

### Example 4

### Expression of Full-Length His₁₀-Tagged Megalin and Cubilin in HEK293 Cells

Expression was performed using the Invitrogen® FreeStyle™ MAX 293 Expression System (cat. no. K9000-10) together with the vector pcDNA™4/HisMax A, B, & C, expressing the recombinant proteins under control of a CMV promoter with an N-terminal His₁₀-tag. cDNA was obtained according to Example 1 using the primers presented in Table 2.

Before starting experiments, cells were established for at least 5 passages. Early-passage cells were used for experiments (below 10 passages). Cultures were divided upon reaching a cell density between 1×10⁶ and 3×10⁶ viable cells/ml (generally every 48-72 hours). Trypan blue exclusion was used to determine cell viability (see below). FreeStyle™ 293 Expression Medium was used as provided. For transfection of suspended FreeStyle™ 293-F cells, we introduced cationic lipid-based FreeStyle™ MAX Reagent, included with the kit. Positive control pCMV SPORT-βgal was provided as a positive control vector for transfection and expression. To transfect a suspension of FreeStyle™ 293-F cells in a 30 ml volume, 37.5 μg of plasmid DNA was added. Approximately 24 h before transfection, the FreeStyle™ 293-F cells were passed at 6−7×10⁵ cells/ml. The flask was placed on an orbital shaker platform rotating at 135 rpm at 37° C., 8% CO₂. On the day of transfection, the cell density was checked, and colonies containing less than 1.2×10⁶ cells/ml were discarded. Next, cells were diluted to 1×10⁶ cells/ml. 30 ml of cells was then added into each 125 ml shake flask. The tube of FreeStyle™ MAX Transfection Reagent was then inverted several times and diluted with 37.5 μg of plasmid DNA in OptiPro™ SFM (Invitrogen) provided in the kit to a total volume of 0.6 ml. In a separate tube, 37.5 μl of FreeStyle™ MAX Reagent in OptiPro™ SFM was also diluted to a total volume of 0.6 ml and mixed gently by rocking. The mixture was incubated gently for 10 minutes at room temperature to allow complexes to form. Then, 1.2 ml of DNA-lipid mixture was added into each 125 ml flask containing cells. The transfected cell cultures were next incubated at 37° C., 8% CO₂ on an orbital shaker platform rotating at 135 rpm for 5 days. Protein expression was detectable within 4-8 h of transfection, with maximal protein yield between 2 and 7 days post-transfection, depending on the construct expressed.

Purification of expressed constructs was done by Ni-affinity and anion exchange FPLC chromatography as described in Smith T et al (2000), Arch Biochem Biophys 375(1):195-200. Pure protein was next eluted in buffer, concentrated using a VivaSpin 20 (Sartorius Stedim Biotech) ultrafiltration spin column at 4° C. and 3 000 g, and finally stored at −80° C.

The expressed proteins were analysed by matrix assisted laser-desorption ionization-time-of-flight (MALDI-TOF) mass spectrometry (MS) using a Bruker Biflex III instrument (Bruker Daltonics) equipped with delayed extraction and reflector. Peptide spectra were internally calibrated using autolytic peptides from trypsin. To identify proteins, searches were performed in the NCBInr sequence database using the ProFound search engine. One miscut, alkylation, and partial oxidation of methionine were allowed. Significance of identification was evaluated according to the probability value, “Z” value, and sequence coverage. The expected m/z of the peptides all appeared as dominant peaks in the mass spectra.

### Example 5

### End-Point Attachment of MEG1 and CUB5-8 onto Beads Using GST

MEG1 and CUB5-8, expressed as described in Example 2, were attached to glutathione-Sephadex® beads as described in Hodneland C D et al, supra. Briefly, 2 mg of glutathione-Sephadex (Sigma) was mixed with 20 μg of purified MEG1, 20 μg of CUB5-8 and 2 ml of 0.25 M NaCl in a glass column and stirred gently for 1 h. Washing was done using buffer (20 mM Tris, pH 7.5+0.25 M NaCl+2 mM EDTA+2 mM EGTA+0.03% Brij-35 (Sigma)).

Successful binding was detected by first washing the column 5 times with PBS (for a total of 5 column volumes), then mixing 1 mg of beads with 0.5 ml of a wash buffer (20 mM Tris+0.25 M NaCl+2 mM EDTA+2 mM EGTA+0.03% Brij-35+20 mM glutathione) and NaOH until pH 8.0 and performing western blots on the supernatant to detect MEG1 and CUB5-8.

### Example 6

### End-Point Attachment of MEG3 and CUB1-7 onto Beads Using Biotin-Avidin Attachment

Biotinylated proteins were synthesized as described in Example 3. Dynabeads® MyOne™ Streptavidin Cl (Invitrogen) were used for coupling according to the manufacturer's instructions. Briefly, after resuspension in PBS and washing 3 times, 10 μg of MEG3 purified protein and 10 μg of CUB1-7 purified protein was added to each mg of beads. The mix was incubated at room temperature for 30 minutes with gentle rotation. A magnet was used to separate the coated beads, which were then washed 6 times in PBS to which was added 0.1% BSA and 0.01% Tween-20. The columns were stored wet until use.

Successful binding was detected by first washing the column 15 times with PBS (total 15 column volumes), then mixing 1 mg of beads with 10 ml of 1.8 mg/ml EDTA pH 8.2 and 95% formamide. After gentle stirring in room temperature for 4 hours, western blots were performed on the supernatant to detect MEG3 and CUB1-7.

### Example 7

### End-Point Attachment of Full-Length Megalin and Cubilin onto Beads Using His₁₀-Tag

Nickel-nitrilotriacetic acid (Ni-NTA) agarose beads (Qiagen) were equilibrated in Tris-buffered saline (TBS) (25 mM Tris-HCl, 137 mM NaCl and 3 mM KCl, pH 7.0) containing 5 mM CaCl₂ and 1 mM MgCl₂, and 10 μg recombinant His-tagged megalin and cubilin proteins expressed as described in Example 4 and prepared in the same buffer were incubated at room temperature for 1 hour. Additional protein was added until binding was saturated (as determined by detecting excess protein in the remaining supernatant). For 1 ml nickel agarose beads, about 12 μg cubilin and megalin were bound, respectively. Protein-coated beads were then washed with 1 ml TBS+20 mM imidazole four times (each for 5 min), and suspended in TBS as a 50% slurry.

Successful binding was detected by first washing the column 15 times (total 15 column volumes) and then mixing 1 mg of beads with 1 ml of 150 mM imidazole and shaking gently for 5 min at room temperature. The supernatant was then analyzed by western blot to detect bound megalin and cubilin.

### Example 8

### Binding of Circulating Peptide Hormones Using Beads Coated with Megalin and Cubilin Polypeptides

Fusion proteins of GST with MEG1 and CUBS-8 or MEG3 and CUB1-7 produced as described in Example 2 were immobilized on glutathione Sepharose® 4B beads at a 1:1 molar ratio of protein as described in Example 5. The beads were then deposited in a 5 ml column (designated “HEP”; 4 ml of beads, dead space 1 ml). A control column was prepared with only Sepharose 4B beads (designated “CTR”; 4 ml of beads, dead space 1 ml). Venous blood (10 ml each) was obtained from a healthy human donor and one patient on maintenance hemodialysis and mixed with dalteparin (Pfizer; 1 IE/ml blood) and spun to remove cells. Of the resulting plasma, one aliquot from each individual was allowed to pass through each column (HEP and CTR) at 1 g. The non-bound fractions were then directly used for two-dimensional gel electrophoresis and the columns subsequently washed 10 times with PBS after which the bound fractions were eluted using 15 ml of elution buffer (50 mM Tris-HCl pH 8 and 10 mM of Reduced Glutathione, Sigma) to each plugged column, which were rocked at room temperature for 20 min, then centrifuged for 5 min at 500 g, drained, and the fluids collected. The elution procedure was repeated a total of three times for each column, and the total eluates of each column pooled for two-dimensional gel electrophoresis.

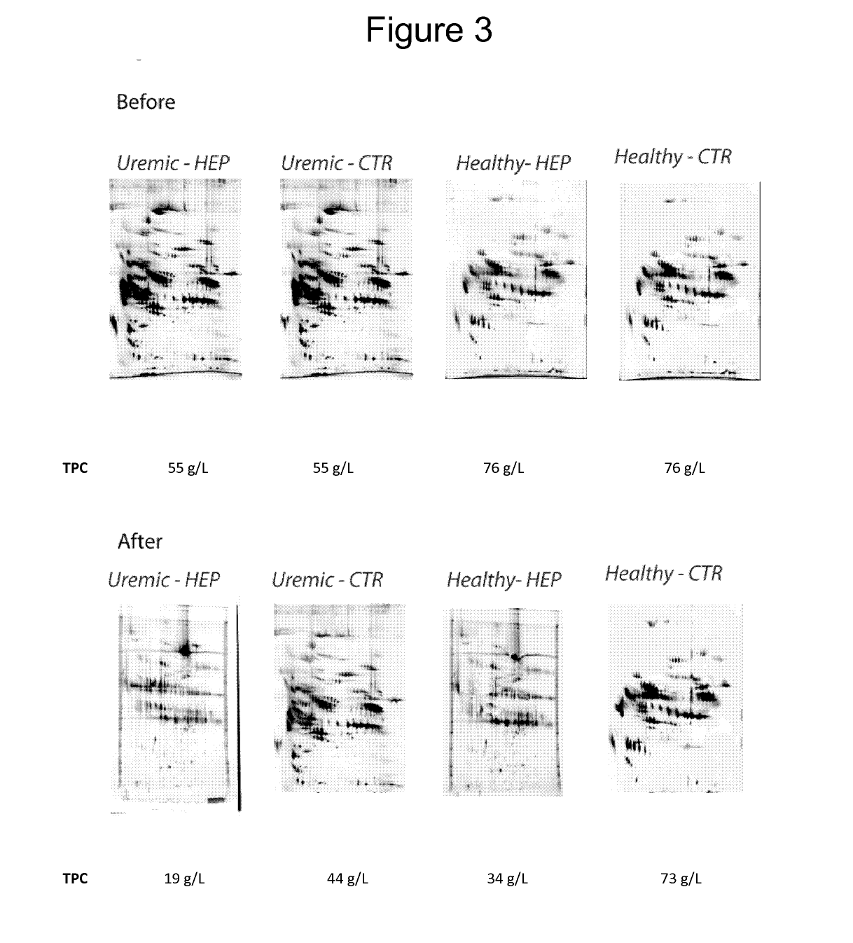

Plasma from before and after passage was tested for total protein content using the Bradford reaction (10 μl fraction+90 μl water+1 ml Bradford reagent (Pierce); absorbance at 590 nm and 450 nm was measured, and the ratio between these used to calculate protein concentration). The same aliquots were then mixed with an equal volume of SDS-PAGE loading buffer containing β-mercaptoethanol and boiled at 100° C. for 5 min. The denatured proteins were then separated on individual 10% SDS-polyacrylamide gels, subsequently stained in silver and Coomassie brilliant blue solutions. The purified proteins were transferred to nitrocellulose membranes (constant current 20 mA at 4° C. overnight) and blocked with 5% milk powder in PBS (room temperature for 2 h). Gels were dried and exposed and scanned in a FujiX2000 phosphoimager (Fuji). Silver stained gels were then scanned in an Image Scanner (Amersham) with the MagicScan32 (Amersham) and AIDA (IMG) softwares and analyzed by the Image Master 2D Elite software (Amersham). Resulting images are shown in FIG. 3.

### Example 9

### Impact of Size Exclusion on Protein Binding

Venous blood (5 ml each) was obtained from seven patients with chronic kidney disease requiring maintenance hemodialysis, pooled and mixed with dalteparin (Pfizer; 1 IE/ml blood). The mixture was then spun down to remove the cells. Of the resulting plasma, one aliquot of 1 ml was allowed to pass through a column with 2 mg of Sephadex® biotinylated to MEG3 and CUB1-7 and prepared according to Example 6. Another aliquot of 1 ml plasma was immediately passed through a size exclusion filter (Centricone 30K, Millipore; spun at 5 000 g for 10 min) and then passed through another column with MEG3 and CUB1-7 prepared according to Example 6.

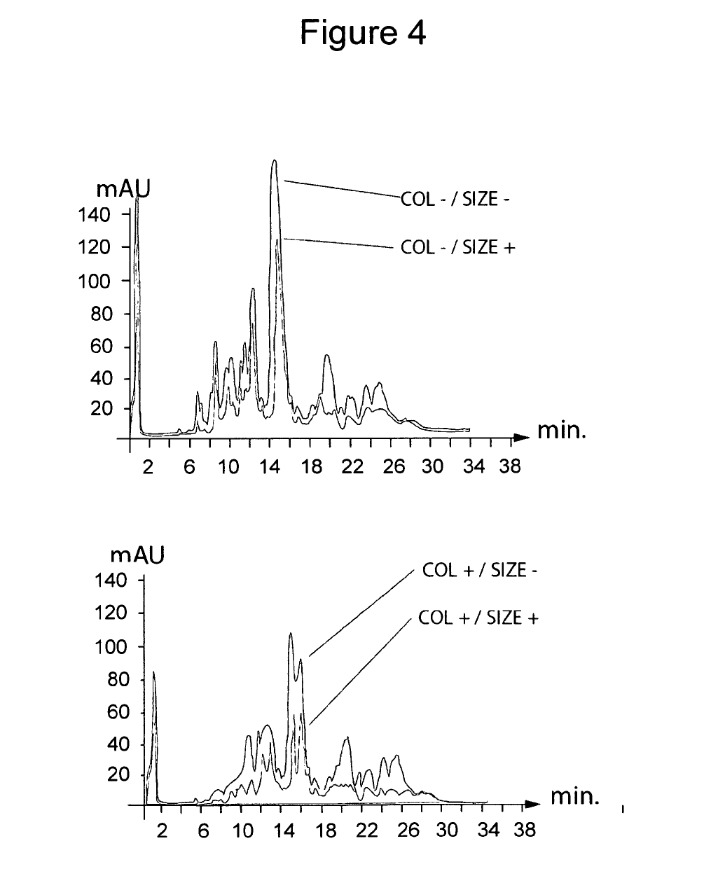

Samples from each of the four groups (with and without size exclusion and before and after the column) were separately analyzed using rpHPLC. 300 μg of plasma was loaded on a mRP-C18 column (Agilent Technologies). Into each sample, urea pellets (22 mg) were added, together with 6 μl neat glacial acetic acid, yielding final concentrations of 6M urea and 0.1% acetic acid. Reverse phase HPLC was performed at a flow rate of 0.75 ml/min and a column temperature of 80° C., with a linear multisegment gradient of buffers A (water/0.1% TFA) and B (acetonitril/0.1% TFA) as follows: time 0 min 3% B; 1 min 3% B; 6 min 30% B; 39 min 55% B; 49 min 100% B; 53 min 100% B; 58 min 3% B. As expected, size exclusion removed a significant amount, but not all, of the larger molecular weight proteins. This removal resulted in significantly better binding of the remaining peptides to the column, perhaps due to less competitive binding. Representative samples of the resulting chromatograms are shown as FIG. 4.

### Example 10

### Impact of Pass-Through Volume and Megalin:Cubilin Ratio on Total Protein Binding

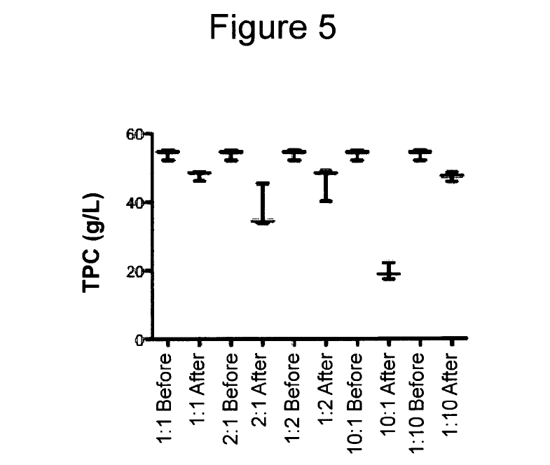

Columns were created as described in Example 5, with the exception that the ratio (w/w) of purified megalin and cubilin domains was varied between 1:10 (1 μg MEG1 and 10 μg CUB5-8/ml of beads) and 10:1 (10 μg MEG1 and 1 μg CUB5-8/ml of beads). Next, venous blood plasma was obtained as in Example 9. One aliquot of 1 ml plasma was allowed to pass through a glass column with 2 mg of surface-immobilized megalin and cubilin beads, while 1 ml of plasma was analyzed immediately. Total protein content (TPC) was determined using the Bradford reaction as described in Example 8. The experiment was repeated three times and mean difference in the change in protein concentrations (after-before aliquots) are given in FIG. 5. Thus, the ratio of 2 mol megalin to 1 mol cubilin reduced the protein in a similar manner to 1 mol megalin to 2 mol cubilin. However, 10 mol megalin to 1 mol cubilin attained a significantly higher level of protein removal during passage through the column.

### Example 11

### Binding of Insulin to MEG5-8 in an Affinity Column

The GST fusion protein MEG5-8 was generated as described in Example 2 and immobilized on glutathione-Sephadex® 4B beads as described in Example 5 (each 2 mg of matrix was mixed with 20 μg of purified MEG5-8). The beads were then deposited in 3 equivalent 5 ml columns (4 ml of beads, dead space 1 ml).

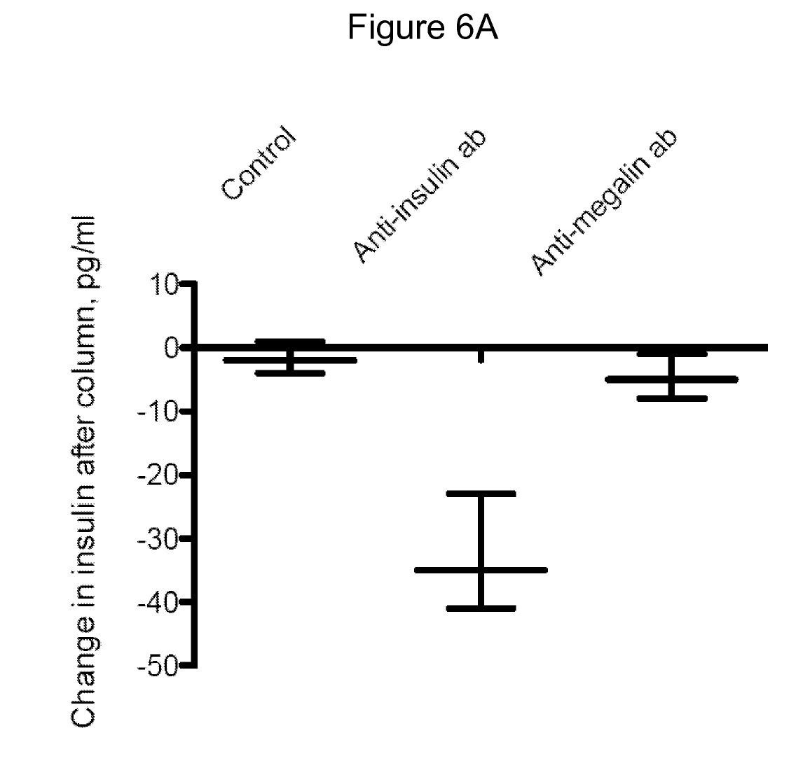

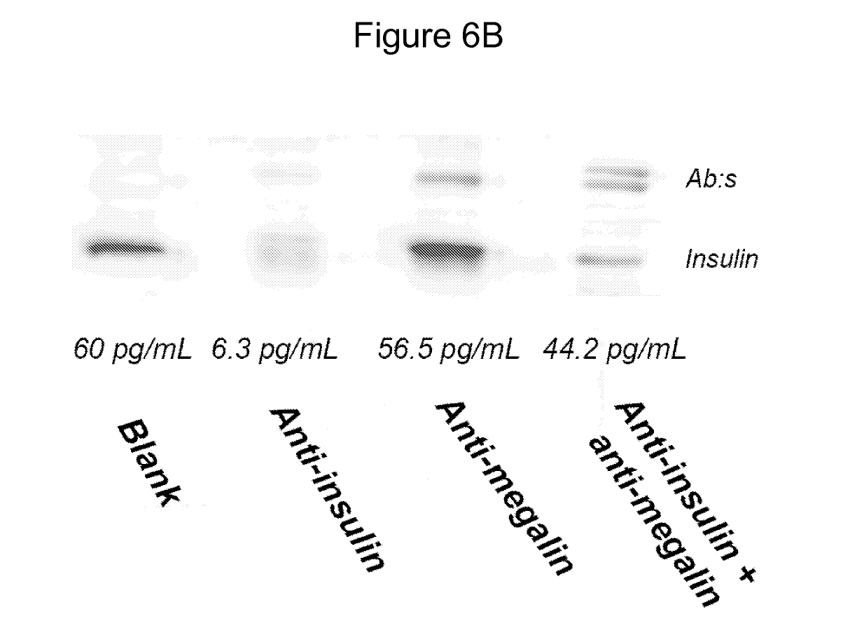

To test the specificity of megalin binding to a ligand, we used 60 μg/ml of purified human insulin (humulin 100 IE/ml; Sanofi-Aventis) mixed with 5% BSA (Sigma) and PBS to a final volume of 5 ml. The mixture was added to a column either on its' own (Blank), mixed with an anti-megalin antibody (Abcam; 20 ng), mixed with an anti-insulin antibody (Millipore; 20 ng) or with both antibodies (20+20 ng). The pass-through fraction was collected and run on a Western blot, a representative sample of which is given in FIG. 6B. We also assessed the insulin concentration in the passed samples using commercial ELISA (Millipore) (see FIG. 6A). As shown in FIG. 6B, anti-megalin, but not anti-insulin, significantly reduced the insulin bound to the column, demonstrating that insulin is captured by our device in a megalin-specific manner.

### Example 12

### Preparation of Full-Length Megalin Column

Full length His₁₀-tagged megalin was prepared as in Example 4, using previously prepared pcDNA™ 4 vectors in Invitrogen® Freestyle™ MAX HEK-293 cells (cat. no. R790-07). Expression was confirmed using Western blot analysis with antibodies kindly donated by Prof. Renata Kozyraki, INSERM, Paris, as previously described (Le Panse et al (1995) Eur J Cell Biol 67(2):120-129; Moestrup et al (1993) J Biol Chem 268:16564-16570).

Nickel-nitrilotriacetic acid (Ni-NTA) agarose beads (Qiagen) were equilibrated in Tris-buffered saline (TBS) (25 mM Tris-HCl, 137 mM NaCl and 3 mM KCl, pH 7.0) containing 5 mM CaCl₂ and 1 mM MgCl₂, and 2 μg recombinant His₁₀-tagged proteins prepared in the same buffer were incubated at room temperature for 1 hour. Additional protein was added until binding was saturated (as determined by detection of excess protein in the remaining supernatant using the Bradford assay). For 1 ml nickel agarose beads, about 10 μg megalin was bound. Protein-coated beads were then washed with 1 ml TBS+20 mM imidazole four times (each for 5 min), and suspended in TBS as a 50% slurry.

Successful binding was detected by first washing the column 15 times (total 15 column volumes) and then mixing 1 mg of beads with 1 ml 150 mM imidazole and shaking gently for 5 min at room temperature. The supernatant was then analyzed by Western blot as above to detect bound megalin.

Finally, 1.5 ml autoclaved glass columns equipped with a 8 μM frit (Whatman® Grade 40 disc filter) were prepared with beads, dried at room temperature and topped up with 20% ethanol while gently shaken. The final columns were capped, sealed and stored at 8° C. until use (within 8 days).

### Example 13

### Treatment of ⅚th Nephrectomized Rats Using Full Length Megalin Columns

Animals:

Male and female Sprague-Dawley rats were obtained from Charles River Labs at 8 weeks of age. The animals were randomly assigned to megalin (MEG) or placebo (CTRL) columns or to sham surgery (SHAM) in a 1:1:1 ratio.

Columns:

Full-length megalin columns were prepared as in Example 12. Placebo columns were prepared in the exact same manner, but using nickel-nitrilotriacetic acid (Ni-NTA) agarose beads (Qiagen) without bound megalin protein.

Animal Handling:

After a 1 week acclimatisation period, 8 male rats were subjected to a 5/6 subtotal nephrectomy as previously described (Shimamura and Morrison (1975) Am J Pathol 79:95-106). Four other male rats were subjected to sham laparectomy without renal parenchymal removal. One animal in each of the nephrectomised groups died during surgery. After surgery, the rats had a 2 week recuperation period with daily handling to reduce experiment stress. Water and standard chow were available ad libitum during the whole trial. The rats were kept in standard cages housing 1 male and 2 females in each. Environmental enrichment was available.

Study Sampling:

The study began on the 15th day post surgery. Rats were anesthetized once per day in the morning using sevoflourane gas inhalation with supplemental O₂ during the whole procedure. Using a tail vein catheter removed after each apheresis and with previous anesthetic ointment, 2 ml blood was drawn into a tube containing 1.1 mg citric acid monohydrate as anticoagulant. The blood was mixed and immediately spun through a 30 kDa cut-off CentriCone® filter (Millipore), all the time keeping in a heated hood at 39° C. The filter was then carefully washed backwards with 0.5 ml isotonic saline pre-heated to 38° C. The size filter pass-through fraction was next passed through a prewashed bead column (MEG or CTRL determined according to study group) and 0.1 ml was collected and stored at −80° C. for later pooled analysis. The remaining treated plasma was mixed with the size filter eluate/non-pass through fraction. The resulting treated blood was immediately reinjected through the catheter and the procedure repeated 3 times. Sham operated animals were exposed to placebo columns (i.e. without bound megalin).

Behavioral Monitoring:

Beginning on study day 20, rats were recorded 24 hours per day for a period of 3 days. Recording was done using IR by a digital video camera (Sony HDR-CX550) linked to a PC. Saved images were reviewed, and the eating frequency and sexual behavior of rats (as described in Sisk and Meek (2001), “Sexual and Reproductive Behaviors” in Current Protocols in Neuroscience, sections 8.2.1-8.2.15) were quantified using the software SBR (Claro et al (1990) Physiol Behav 48(3):489-493).

Results:

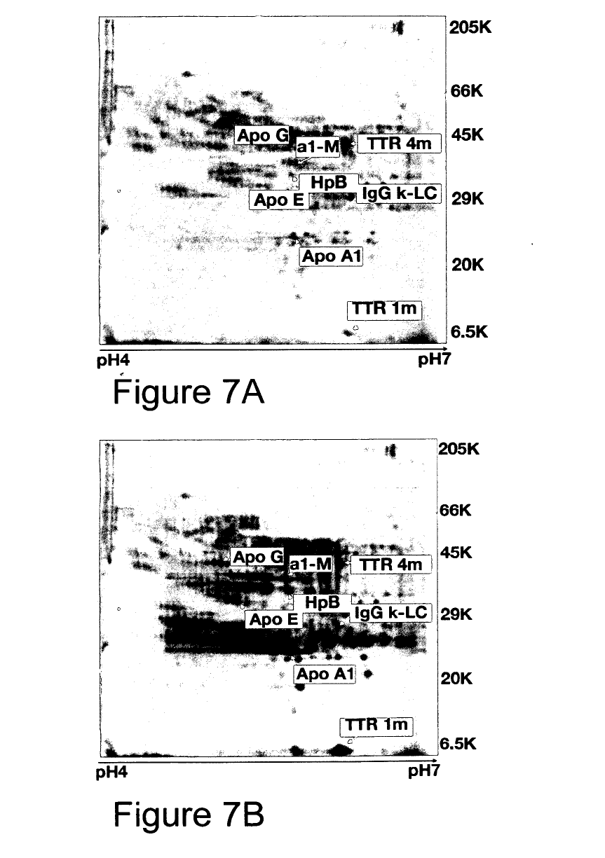

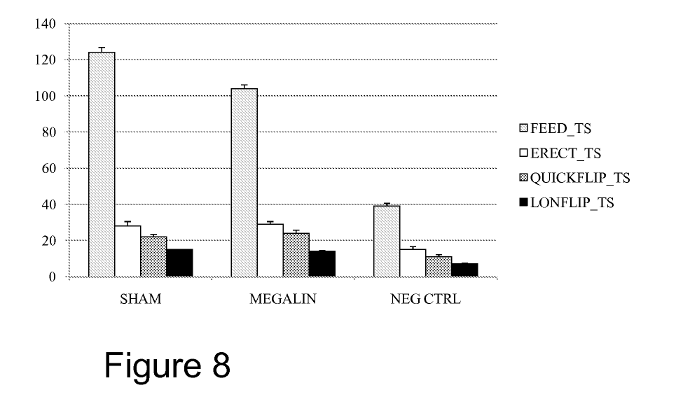

Gels and protein content of pooled treated fractions from 24 column passes over 8 days are shown in FIG. 7. Compared to the SHAM group with normal renal function, nephrectomised rats had significantly higher levels of serum creatinine (SHAM 0.8±0.1 vs. MEG 1.8±0.4 and CTRL 1.5±0.4 mg/dl; p<0.05 for SHAM vs. the other groups) and urea (22.7±6.1 vs. 45.0±5.7 and 57.8±8.2 mg/dl respectively; p<0.05). Furthermore, as shown in FIG. 8, while both sexual and feeding behaviors were significantly reduced in CTRL rats, but not in MEG rats, as compared to SHAM non-nephrectomized rats. The differences between SHAM and CTRL were all significant, while the differences between SHAM and MEG were not. Feeding, erection and quickflip behaviors were significantly (p<0.05) more common in MEG rats than in CTRL rats, while the difference in longflip behavior was not statistically significant between these two groups.

1. A separation medium comprising either:
a. at least one megalin polypeptide, or
b. at least one cubilin polypeptide,
immobilized on a support.

2. A separation medium according to claim 1, wherein the medium comprises at least one megalin polypeptide and at least one cubilin polypeptide immobilized on a support.

3. (canceled)

4. A separation medium according to claim 1, wherein said megalin polypeptide, when present, has an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12, and amino acid sequences having at least 80% identity therewith.

5. A separation medium according to anyone of claims 1, 3 and 4, wherein said cubilin polypeptide, when present, has an amino acid sequence selected from the group consisting of SEQ ID NO:13, SEQ ID NO:14, SEQ ID NO:15, SEQ ID NO:16, SEQ ID NO:17, SEQ ID NO:18, SEQ ID NO:19 and SEQ ID NO:20, and amino acid sequences having at least 80% identity therewith.

6. A medical device for extracorporeal treatment of a complex biological fluid, comprising a separation medium according to claim 1.

7. A medical device according to claim 6, wherein said complex biological fluid is blood.

8. A medical device according to claim 6, wherein said complex biological fluid comprises a low molecular weight protein.

9. A medical device according to claim 8, wherein said low molecular weight protein is selected from the group consisting of peptide hormones, enzymes, immunoglobulin light chains, myoglobulin and vitamin-binding proteins.

10. A medical device according to claim 6, wherein said device comprises a size filter.

11. A medical device according to claim 6, wherein said device comprises a charge filter.

12. A dialysis device for extracorporeal treatment of a complex biological fluid, comprising a medical device according to claim 6.

13. Method for extracorporeal removal of a low molecular weight protein, or fragment or derivative thereof, from a complex biological fluid, comprising the steps:
a) providing a sample of complex biological fluid containing a low molecular weight protein, fragment, or derivative thereof, that has a binding affinity for megalin and/or cubilin,
b) bringing said sample into contact with either a separation medium according to claim 1 or a device according to claim 6, under conditions allowing binding of said low molecular weight protein, fragment, or derivative thereof, to said at least one megalin polypeptide or said at least one cubilin polypeptide,
c) separating said sample from said support, such that at least part of the total amount of said low molecular weight protein, fragment, or derivative thereof, initially present in said sample is retained on the support, and
d) recovering said sample containing a reduced amount of said low molecular weight protein, fragment, or derivative thereof.

14. Method according to claim 13, further comprising subjecting the sample to a size filtration step, whereby high molecular weight components are removed from the sample before performing step b).

15. Method according to claim 13, further comprising subjecting the sample to a charge filtration step, whereby components having a pI of no more than 8 are removed from the sample before performing step b).

16. Method according to claim 13, wherein said method further comprises a step e), wherein said retained low molecular weight protein, fragment, or derivative thereof, is eluted.

17. Method for treatment of a mammalian subject suffering from a condition caused or aggravated by a low molecular weight protein, or fragment or derivative thereof, comprising the steps of:
a) extracting blood from the subject,
b) using a method according to claim 13, such that at least part of the total amount of said low molecular weight protein, fragment or derivative thereof, initially present in said extracted blood is retained on the support, and
c) reintroducing the blood, containing a reduced amount of said low molecular weight protein, fragment, or derivative thereof, into the subject.

18. (canceled)

19. The separation medium of claim 1, wherein the support is made from a material selected from the group consisting of glass, cellulose, cellulose acetate, chitin, chitosan, cross-linked dextran, cross-linked agarose, agar gel support, polypropylene, polyethylene, polysulfone, polyacrylonitrile, polytetrafluoroethylene, polystyrene, polyurethane, silicone and amylose coated particles.

20. The separation medium of claim 2, wherein the molar ratio between said at least one megalin polypeptide and said at least one cubilin polypeptide is from 1:100 to 100:1.

21. The method of claim 13, wherein the complex biological fluid is blood, plasma, or urine.

22. A method of performing hemodialysis, hemofiltration, or hemodiafiltration, comprising:
connecting the device of claim 6 to an extracorporeal blood circuit; and
using the device of claim 6 to remove low molecular weight protein from the blood.


SEQUENCE LISTING (20 sequences)

SEQ ID NO: 1 - protein, 4655 aa, Homo sapiens
MDRGPAAVACTLLLALVACLAPASGQECDSAHFRCGSGHCIPADWRCDGTKDCSDDADEIGCAVVTCQQGYFKCQSEGQCIPNSWVCDQDQDCDDGSDERQDCSQSTCSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDEHACNYPTCGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCKANDSFGEASIIFSNGRDLLIGDIHGRSFRILVESQNRGVAVGVAFHYHLQRVFWTDTVQNKVFSVDINGLNIQEVLNVSVETPENLAVDWVNNKIYLVETKVNRIDMVNLDGSYRVTLITENLGHPRGIAVDPTVGYLFFSDWESLSGEPKLERAFMDGSNRKDLVKTKLGWPAGVTLDMISKRVYWVDSRFDYIETVTYDGIQRKTVVHGGSLIPHPFGVSLFEGQVFFTDWTKMAVLKANKFTETNPQVYYQASLRPYGVTVYHSLRQPYATNPCKDNNGGCEQVCVLSHRTDNDGLGFRCKCTFGFQLDTDERHCIAVQNFLIFSSQVAIRGIPFTLSTQEDVMVPVSGNPSFFVGIDFDAQDSTIFFSDMSKHMIFKQKIDGTGREILAANRVENVESLAFDWISKNLYWTDSHYKSISVMRLADKTRRTVVQYLNNPRSVVVHPFAGYLFFTDWFRPAKIMRAWSDGSHLLPVINTTLGWPNGLAIDWAASRLYWVDAYFDKIEHSTFDGLDRRRLGHIEQMTHPFGLAIFGEHLFFTDWRLGAIIRVRKADGGEMTVIRSGIAYILHLKSYDVNIQTGSNACNQPTHPNGDCSHFCFPVPNFQRVCGCPYGMRLASNHLTCEGDPTNEPPTEQCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCKVTASESLLLLVASQNKIIADSVTSQVHNIYSLVENGSYIVAVDFDSISGRIFWSDATQGKTWSAFQNGTDRRVVFDSSIILTETIAIDWVGRNLYWTDYALETIEVSKIDGSHRTVLISKNLTNPRGLALDPRMNEHLLFWSDWGHHPRIERASMDGSMRTVIVQDKIFWPCGLTIDYPNRLLYFMDSYLDYMDFCDYNGHHRRQVIASDLIIRHPYALTLFEDSVYWTDRATRRVMRANKWHGGNQSVVMYNIQWPLGIVAVHPSKQPNSVNPCAFSRCSHLCLLSSQGPHFYSCVCPSGWSLSPDLLNCLRDDQPFLITVRQHIIFGISLNPEVKSNDAMVPIAGIQNGLDVEFDDAEQYIYWVENPGEIHRVKTDGTNRTVFASISMVGPSMNLALDWISRNLYSTNPRTQSIEVLTLHGDIRYRKTLIANDGTALGVGFPIGITVDPARGKLYWSDQGTDSGVPAKIASANMDGTSVKTLFTGNLEHLECVTLDIEEQKLYWAVTGRGVIERGNVDGTDRMILVHQLSHPWGIAVHDSFLYYTDEQYEVIERVDKATGANKIVLRDNVPNLRGLQVYHRRNAAESSNGCSNNMNACQQICLPVPGGLFSCACATGFKLNPDNRSCSPYNSFIVVSMLSAIRGFSLELSDHSETMVPVAGQGRNALHVDVDVSSGFIYWCDFSSSVASDNAIRRIKPDGSSLMNIVTHGIGENGVRGIAVDWVAGNLYFTNAFVSETLIEVLRINTTYRRVLLKVTVDMPRHIVVDPKNRYLFWADYGQRPKIERSFLDCTNRTVLVSEGIVTPRGLAVDRSDGYVYWVDDSLDIIARIRINGENSEVIRYGSRYPTPYGITVFENSIIWVDRNLKKIFQASKEPENTEPPTVIRDNINWLRDVTIFDKQVQPRSPAEVNNNPCLENNGGCSHLCFALPGLHTPKCDCAFGTLQSDGKNCAISTENFLIFALSNSLRSLHLDPENHSPPFQTINVERTVMSLDYDSVSDRIYFTQNLASGVGQISYATLSSGIHTPTVIASGIGTADGIAFDWITRRIYYSDYLNQMINSMAEDGSNRTVIARVPKPRAIVLDPCQGYLYWADWDTHAKIERATLGGNFRVPIVNSSLVMPSGLTLDYEEDLLYWVDASLQRIERSTLTGVDREVIVNAAVHAFGLTLYGQYIYWTDLYTQRIYRANKYDGSGQIAMTTNLLSQPRGINTVVKNQKQQCNNPCEQFNGGCSHICAPGPNGAECQCPHEGNWYLANNRKHCIVDNGERCGASSFTCSNGRCISEEWKCDNDNDCGDGSDEMESVCALHTCSPTAFTCANGRCVQYSYRCDYYNDCGDGSDEAGCLFRDCNATTEFMCNNRRCIPREFICNGVDNCHDNNTSDEKNCPDRTCQSGYTKCHNSNICIPRVYLCDGDNDCGDNSDENPTYCTTHTCSSSEFQCASGRCIPQHWYCDQETDCFDASDEPASCGHSERTCLADEFKCDGGRCIPSEWICDGDNDCGDMSDEDKRHQCQNQNCSDSEFLCVNDRPPDRRCIPQSWVCDGDVDCTDGYDENQNCTRRTCSENEFTCGYGLCIPKIFRCDRHNDCGDYSDERGCLYQTCQQNQFTCQNGRCISKTFVCDEDNDCGDGSDELMHLCHTPEPTCPPHEFKCDNGRCIEMMKLCNHLDDCLDNSDEKGCGINECHDPSISGCDHNCTDTLTSFYCSCRPGYKLMSDKRTCVDIDECTEMPFVCSQKCENVIGSYICKCAPGYLREPDGKTCRQNSNIEPYLIFSNRYYLRNLTIDGYFYSLILEGLDNVVALDFDRVEKRLYWIDTQRQVIERMFLNKTNKETIINHRLPAAESLAVDWVSRKLYWLDARLDGLFVSDLNGGHRRMLAQHCVDANNTFCFDNPRGLALHPQYGYLYWADWGHRAYIGRVGMDGTNKSVIISTKLEWPNGITIDYTNDLLYWADAHLGYIEYSDLEGHHRHTVYDGALPHPFAITIFEDTIYWTDWNTRTVEKGNKYDGSNRQTLVNTTHRPFDIHVYHPYRQPIVSNPCGTNNGGCSHLCLIKPGGKGFTCECPDDFRTLQLSGSTYCMPMCSSTQFLCANNEKCIPIWWKCDGQKDCSDGSDELALCPQRFCRLGQFQCSDGNCTSPQTLCNAHQNCPDGSDEDRLLCENHHCDSNEWQCANKRCIPESWQCDTFNDCEDNSDEDSSHCASRTCRPGQFRCANGRCIPQAWKCDVDNDCGDHSDEPIEECMSSAHLCDNFTEFSCKTNYRCIPKWAVCNGVDDCRDNSDEQGCEERTCHPVGDFRCKNHHCIPLRWQCDGQNDCGDNSDEENCAPRECTESEFRCVNQQCIPSRWICDHYNDCGDNSDERDCEMRTCHPEYFQCTSGHCVHSELKCDGSADCLDASDEADCPTRFPDGAYCQATMFECKNHVCIPPYWKCDGDDDCGDGSDEELHLCLDVPCNSPNRFRCDNNRCIYSHEVCNGVDDCGDGTDETEEHCRKPTPKPCTEYEYKCGNGHCIPHDNVCDDADDCGDWSDELGCNKGKERTCAENICEQNCTQLNEGGFICSCTAGFETNVFDRTSCLDINECEQFGTCPQHCRNTKGSYECVCADGFTSMSDRPGKRCAAEGSSPLLLLPDNVRIRKYNLSSERFSEYLQDEEYIQAVDYDWDPKDIGLSVVYYTVRGEGSRFGAIKRAYIPNFESGRNNLVQEVDLKLKYVMQPDGIAVDWVGRHIYWSDVKNKRIEVAKLDGRYRKWLISTDLDQPAAIAVNPKLGLMFWTDWGKEPKIESAWMNGEDRNILVFEDLGWPTGLSIDYLNNDRIYWSDFKEDVIETIKYDGTDRRVIAKEAMNPYSLDIFEDQLYWISKEKGEVWKQNKFGQGKKEKTLVVNPWLTQVRIFHQLRYNKSVPNLCKQICSHLCLLRPGGYSCACPQGSSFIEGSTTECDAAIELPINLPPPCRCMHGGNCYFDETDLPKCKCPSGYTGKYCEMAFSKGISPGTTAVAVLLTILLIVVIGALAIAGFFHYRRTGSLLPALPKLPSLSSLVKPSENGNGVTFRSGADLNMDIGVSGFGPETAIDRSMAMSEDFVMEMGKQPIIFENPMYSARDSAVKVVQPIQVTVSENVDNKNYGSPINPSEIVPETNPTSPAADGTQVTKWNLFKRKSKQTTNFENPIYAQMENEQKESVAATPPPSPSLPAKPKPPSRRDPTPTYSATEDTFKDTANLVKEDSEV

SEQ ID NO: 2 - protein, 474 aa, Homo sapiens
CDSAHFRCGSGHCIPADWRCDGTKDCSDDADEIGCAVVTCQQGYFKCQSEGQCIPNSWVCDQDQDCDDGSDERQDCSQSTCSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDEHACNYPTCGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCKANDSFGEASIIFSNGRDLLIGDIHGRSFRILVESQNRGVAVGVAFHYHLQRVFWTDTVQNKVFSVDINGLNIQEVLNVSVETPENLAVDWVNNKIYLVETKVNRIDMVNLDGSYR

SEQ ID NO: 3 - protein, 1493 aa, Homo sapiens
VLNVSVETPENLAVDWVNNKIYLVETKVNRIDMVNLDGSYRVTLITENLGHPRGIAVDPTVGYLFFSDWESLSGEPKLERAFMDGSNRKDLVKTKLGWPAGVTLDMISKRVYWVDSRFDYIETVTYDGIQRKTVVHGGSLIPHPFGVSLFEGQVFFTDWTKMAVLKANKFTETNPQVYYQASLRPYGVTVYHSLRQPYATNPCKDNNGGCEQVCVLSHRTDNDGLGFRCKCTFGFQLDTDERHCIAVQNFLIFSSQVAIRGIPFTLSTQEDVMVPVSGNPSFFVGIDFDAQDSTIFFSDMSKHMIFKQKIDGTGREILAANRVENVESLAFDWISKNLYWTDSHYKSISVMRLADKTRRTVVQYLNNPRSVVVHPFAGYLFFTDWFRPAKIMRAWSDGSHLLPVINTTLGWPNGLAIDWAASRLYWVDAYFDKIEHSTFDGLDRRRLGHIEQMTHPFGLAIFGEHLFFTDWRLGAIIRVRKADGGEMTVIRSGIAYILHLKSYDVNIQTGSNACNQPTHPNGDCSHFCFPVPNFQRVCGCPYGMRLASNHLTCEGDPTNEPPTEQCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCKVTASESLLLLVASQNKIIADSVTSQVHNIYSLVENGSYIVAVDFDSISGRIFWSDATQGKTWSAFQNGTDRRVVFDSSIILTETIAIDWVGRNLYWTDYALETIEVSKIDGSHRTVLISKNLTNPRGLALDPRMNEHLLFWSDWGHHPRIERASMDGSMRTVIVQDKIFWPCGLTIDYPNRLLYFMDSYLDYMDFCDYNGHHRRQVIASDLIIRHPYALTLFEDSVYWTDRATRRVMRANKWHGGNQSVVMYNIQWPLGIVAVHPSKQPNSVNPCAFSRCSHLCLLSSQGPHFYSCVCPSGWSLSPDLLNCLRDDQPFLITVRQHIIFGISLNPEVKSNDAMVPIAGIQNGLDVEFDDAEQYIYWVENPGEIHRVKTDGTNRTVFASISMVGPSMNLALDWISRNLYSTNPRTQSIEVLTLHGDIRYRKTLIANDGTALGVGFPIGITVDPARGKLYWSDQGTDSGVPAKIASANMDGTSVKTLFTGNLEHLECVTLDIEEQKLYWAVTGRGVIERGNVDGTDR

SEQ ID NO: 4 - protein, 855 aa, Homo sapiens
IVNSSLVMPSGLTLDYEEDLLYWVDASLQRIERSTLTGVDREVIVNAAVHAFGLTLYGQYIYWTDLYTQRIYRANKYDGSGQIAMTTNLLSQPRGINTVVKNQKQQCNNPCEQFNGGCSHICAPGPNGAECQCPHEGNWYLANNRKHCIVDNGERCGASSFTCSNGRCISEEWKCDNDNDCGDGSDEMESVCALHTCSPTAFTCANGRCVQYSYRCDYYNDCGDGSDEAGCLFRDCNATTEFMCNNRRCIPREFICNGVDNCHDNNTSDEKNCPDRTCQSGYTKCHNSNICIPRVYLCDGDNDCGDNSDENPTYCTTHTCSSSEFQCASGRCIPQHWYCDQETDCFDASDEPASCGHSERTCLADEFKCDGGRCIPSEWICDGDNDCGDMSDEDKRHQCQNQNCSDSEFLCVNDRPPDRRCIPQSWVCDGDVDCTDGYDENQNCTRRTCSENEFTCGYGLCIPKIFRCDRHNDCGDYSDERGCLYQTCQQNQFTCQNGRCISKTFVCDEDNDCGDGSDELMHLCHTPEPTCPPHEFKCDNGRCIEMMKLCNHLDDCLDNSDEKGCGINECHDPSISGCDHNCTDTLTSFYCSCRPGYKLMSDKRTCVDIDECTEMPFVCSQKCENVIGSYICKCAPGYLREPDGKTCRQNSNIEPYLIFSNRYYLRNLTIDGYFYSLILEGLDNVVALDFDRVEKRLYWIDTQRQVIERMFLNKTNKETIINHRLPAAESLAVDWVSRKLYWLDARLDGLFVSDLNGGHRRMLAQHCVDANNTFCFDNPRGLALHPQYGYLYWADWGHRAYIGRVGMDGTNKSVIISTKLEWPNGITIDYTNDLLYWADAHLGYIEYSDLEGHHR

SEQ ID NO: 5 - protein, 1248 aa, Homo sapiens
PHPFAITIFEDTIYWTDWNTRTVEKGNKYDGSNRQTLVNTTHRPFDIHVYHPYRQPIVSNPCGTNNGGCSHLCLIKPGGKGFTCECPDDFRTLQLSGSTYCMPMCSSTQFLCANNEKCIPIWWKCDGQKDCSDGSDELALCPQRFCRLGQFQCSDGNCTSPQTLCNAHQNCPDGSDEDRLLCENHHCDSNEWQCANKRCIPESWQCDTFNDCEDNSDEDSSHCASRTCRPGQFRCANGRCIPQAWKCDVDNDCGDHSDEPIEECMSSAHLCDNFTEFSCKTNYRCIPKWAVCNGVDDCRDNSDEQGCEERTCHPVGDFRCKNHHCIPLRWQCDGQNDCGDNSDEENCAPRECTESEFRCVNQQCIPSRWICDHYNDCGDNSDERDCEMRTCHPEYFQCTSGHCVHSELKCDGSADCLDASDEADCPTRFPDGAYCQATMFECKNHVCIPPYWKCDGDDDCGDGSDEELHLCLDVPCNSPNRFRCDNNRCIYSHEVCNGVDDCGDGTDETEEHCRKPTPKPCTEYEYKCGNGHCIPHDNVCDDADDCGDWSDELGCNKGKERTCAENICEQNCTQLNEGGFICSCTAGFETNVFDRTSCLDINECEQFGTCPQHCRNTKGSYECVCADGFTSMSDRPGKRCAAEGSSPLLLLPDNVRIRKYNLSSERFSEYLQDEEYIQAVDYDWDPKDIGLSVVYYTVRGEGSRFGAIKRAYIPNFESGRNNLVQEVDLKLKYVMQPDGIAVDWVGRHIYWSDVKNKRIEVAKLDGRYRKWLISTDLDQPAAIAVNPKLGLMFWTDWGKEPKIESAWMNGEDRNILVFEDLGWPTGLSIDYLNNDRIYWSDFKEDVIETIKYDGTDRRVIAKEAMNPYSLDIFEDQLYWISKEKGEVWKQNKFGQGKKEKTLVVNPWLTQVRIFHQLRYNKSVPNLCKQICSHLCLLRPGGYSCACPQGSSFIEGSTTECDAAIELPINLPPPCRCMHGGNCYFDETDLPKCKCPSGYTGKYCEMAFSKGISPGTTAVAVLLTILLIVVIGALAIAGFFHYRRTGSLLPALPKLPSLSSLVKPSENGNGVTFRSGADLNMDIGVSGFGPETAIDRSMAMSEDFVMEMGKQPIIFENPMYSARDSAVKVVQPIQVTVSENVDNKNYGSPINPSEIVPETNPTSPAADGTQVTKWNLFKRKSKQTTNFENPIYAQMENEQKESVAATPPPSPSLPAKPKPPSRRDPTPTYSATEDTFKDTANLVKEDSEV

SEQ ID NO: 6 - protein, 32 aa, Homo sapiens
CDSAHFRCGSGHCIPADWRCDGTKDCSDDADE

SEQ ID NO: 7 - protein, 107 aa, Homo sapiens
CSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDE

SEQ ID NO: 8 - protein, 683 aa, Homo sapiens
CGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCKANDSFGEASIIFSNGRDLLIGDIHGRSFRILVESQNRGVAVGVAFHYHLQRVFWTDTVQNKVFSVDINGLNIQEVLNVSVETPENLAVDWVNNKIYLVETKVNRIDMVNLDGSYRVTLITENLGHPRGIAVDPTVGYLFFSDWESLSGEPKLERAFMDGSNRKDLVKTKLGWPAGVTLDMISKRVYWVDSRFDYIETVTYDGIQRKTVVHGGSLIPHPFGVSLFEGQVFFTDWTKMAVLKANKFTETNPQVYYQASLRPYGVTVYHSLRQPYATNPCKDNNGGCEQVCVLSHRTDNDGLGFRCKCTFGFQLDTDERHCIAVQNFLIFSSQVAIRGIPFTLSTQEDVMVPVSGNPSFFVGIDFDAQDSTIFFSDMSKHMIFKQKIDGTGREILAANRVENVESLAFDWISKNLYWTDSHYKSISVMRLADKTRRTVVQYLNNPRSVVVHPFAGYLFFTDWFRPAKIMRAWSDGSHLLPVINTTLGWPNGLAIDWAASRLYWVDAYFDKIEHSTFDGLDR

SEQ ID NO: 9 - protein, 1518 aa, Homo sapiens
QCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCKVTASESLLLLVASQNKIIADSVTSQVHNIYSLVENGSYIVAVDFDSISGRIFWSDATQGKTWSAFQNGTDRRVVFDSSIILTETIAIDWVGRNLYWTDYALETIEVSKIDGSHRTVLISKNLTNPRGLALDPRMNEHLLFWSDWGHHPRIERASMDGSMRTVIVQDKIFWPCGLTIDYPNRLLYFMDSYLDYMDFCDYNGHHRRQVIASDLIIRHPYALTLFEDSVYWTDRATRRVMRANKWHGGNQSVVMYNIQWPLGIVAVHPSKQPNSVNPCAFSRCSHLCLLSSQGPHFYSCVCPSGWSLSPDLLNCLRDDQPFLITVRQHIIFGISLNPEVKSNDAMVPIAGIQNGLDVEFDDAEQYIYWVENPGEIHRVKTDGTNRTVFASISMVGPSMNLALDWISRNLYSTNPRTQSIEVLTLHGDIRYRKTLIANDGTALGVGFPIGITVDPARGKLYWSDQGTDSGVPAKIASANMDGTSVKTLFTGNLEHLECVTLDIEEQKLYWAVTGRGVIERGNVDGTDRMILVHQLSHPWGIAVHDSFLYYTDEQYEVIERVDKATGANKIVLRDNVPNLRGLQVYHRRNAAESSNGCSNNMNACQQICLPVPGGLFSCACATGFKLNPDNRSCSPYNSFIVVSMLSAIRGFSLELSDHSETMVPVAGQGRNALHVDVDVSSGFIYWCDFSSSVASDNAIRRIKPDGSSLMNIVTHGIGENGVRGIAVDWVAGNLYFTNAFVSETLIEVLRINTTYRRVLLKVTVDMPRHIVVDPKNRYLFWADYGQRPKIERSFLDCTNRTVLVSEGIVTPRGLAVDRSDGYVYWVDDSLDIIARIRINGENSEVIRYGSRYPTPYGITVFENSIIWVDRNLKKIFQASKEPENTEPPTVIRDNINWLRDVTIFDKQVQPRSPAEVNNNPCLENNGGCSHLCFALPGLHTPKCDCAFGTLQSDGKNCAISTENFLIFALSNSLRSLHLDPENHSPPFQTINVERTVMSLDYDSVSDRIYFTQNLASGVGQISYATLSSGIHTPTVIASGIGTADGIAFDWITRRIYYSDYLNQMINSMAEDGSNRTVIARVPKPRAIVLDPCQGYLYWADWDTHAKIERATLGGNFR

SEQ ID NO: 10 - protein, 565 aa, Homo sapiens
IVNSSLVMPSGLTLDYEEDLLYWVDASLQRIERSTLTGVDREVIVNAAVHAFGLTLYGQYIYWTDLYTQRIYRANKYDGSGQIAMTTNLLSQPRGINTVVKNQKQQCNNPCEQFNGGCSHICAPGPNGAECQCPHEGNWYLANNRKHCIVDNGERCGASSFTCSNGRCISEEWKCDNDNDCGDGSDEMESVCALHTCSPTAFTCANGRCVQYSYRCDYYNDCGDGSDEAGCLFRDCNATTEFMCNNRRCIPREFICNGVDNCHDNNTSDEKNCPDRTCQSGYTKCHNSNICIPRVYLCDGDNDCGDNSDENPTYCTTHTCSSSEFQCASGRCIPQHWYCDQETDCFDASDEPASCGHSERTCLADEFKCDGGRCIPSEWICDGDNDCGDMSDEDKRHQCQNQNCSDSEFLCVNDRPPDRRCIPQSWVCDGDVDCTDGYDENQNCTRRTCSENEFTCGYGLCIPKIFRCDRHNDCGDYSDERGCLYQTCQQNQFTCQNGRCISKTFVCDEDNDCGDGSDELMHLCHTPEPTCPPHEFKCDNGRCIEMMKLCNHLDDCLDNSDEKGC

SEQ ID NO: 11 - protein, 1504 aa, Homo sapiens
DIDECTEMPFVCSQKCENVIGSYICKCAPGYLREPDGKTCRQNSNIEPYLIFSNRYYLRNLTIDGYFYSLILEGLDNVVALDFDRVEKRLYWIDTQRQVIERMFLNKTNKETIINHRLPAAESLAVDWVSRKLYWLDARLDGLFVSDLNGGHRRMLAQHCVDANNTFCFDNPRGLALHPQYGYLYWADWGHRAYIGRVGMDGTNKSVIISTKLEWPNGITIDYTNDLLYWADAHLGYIEYSDLEGHHRHTVYDGALPHPFAITIFEDTIYWTDWNTRTVEKGNKYDGSNRQTLVNTTHRPFDIHVYHPYRQPIVSNPCGTNNGGCSHLCLIKPGGKGFTCECPDDFRTLQLSGSTYCMPMCSSTQFLCANNEKCIPIWWKCDGQKDCSDGSDELALCPQRFCRLGQFQCSDGNCTSPQTLCNAHQNCPDGSDEDRLLCENHHCDSNEWQCANKRCIPESWQCDTFNDCEDNSDEDSSHCASRTCRPGQFRCANGRCIPQAWKCDVDNDCGDHSDEPIEECMSSAHLCDNFTEFSCKTNYRCIPKWAVCNGVDDCRDNSDEQGCEERTCHPVGDFRCKNHHCIPLRWQCDGQNDCGDNSDEENCAPRECTESEFRCVNQQCIPSRWICDHYNDCGDNSDERDCEMRTCHPEYFQCTSGHCVHSELKCDGSADCLDASDEADCPTRFPDGAYCQATMFECKNHVCIPPYWKCDGDDDCGDGSDEELHLCLDVPCNSPNRFRCDNNRCIYSHEVCNGVDDCGDGTDETEEHCRKPTPKPCTEYEYKCGNGHCIPHDNVCDDADDCGDWSDELGCNKGKERTCAENICEQNCTQLNEGGFICSCTAGFETNVFDRTSCLDINECEQFGTCPQHCRNTKGSYECVCADGFTSMSDRPGKRCAAEGSSPLLLLPDNVRIRKYNLSSERFSEYLQDEEYIQAVDYDWDPKDIGLSVVYYTVRGEGSRFGAIKRAYIPNFESGRNNLVQEVDLKLKYVMQPDGIAVDWVGRHIYWSDVKNKRIEVAKLDGRYRKWLISTDLDQPAAIAVNPKLGLMFWTDWGKEPKIESAWMNGEDRNILVFEDLGWPTGLSIDYLNNDRIYWSDFKEDVIETIKYDGTDRRVIAKEAMNPYSLDIFEDQLYWISKEKGEVWKQNKFGQGKKEKTLVVNPWLTQVRIFHQLRYNKSVPNLCKQICSHLCLLRPGGYSCACPQGSSFIEGSTTECDAAIELPINLPPPCRCMHGGNCYFDETDLPKCKCPSGYTGKYCEMAFSKGISPGTTAVAVLLTILLIVVIGALAIAGFFHYRRTGSLLPALPKLPSLSSLVKPSENGNGVTFRSGADLNMDIGVSGFGPETAIDRSMAMSEDFVMEMGKQPIIFENPMYSARDSAVKVVQPIQVTVSENVDNKNYGSPINPSEIVPETNPTSPAADGTQVTKWNLFKRKSKQTTNFENPIYAQMENEQKESVAATPPPSPSLPAKPKPPSRRDPTPTYSATEDTFKDTANLVKEDSEV

SEQ ID NO: 12 - protein, 2515 aa, Homo sapiens
CDSAHFRCGSGHCIPADWRCDGTKDCSDDADEIGCAVVTCQQGYFKCQSEGQCIPNSWVCDQDQDCDDGSDERQDCSQSTCSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDEHACNYPTCGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCKANDSFGEASIIFSNGRDLLIGDIHGRSFRILVESQNRGVAVGVAFHYHLQRVFWTDTVQNKVFSVDINGLNIQEVLNVSVETPENLAVDWVNNKIYLVETKVNRIDMVNLDGSYRVTLITENLGHPRGIAVDPTVGYLFFSDWESLSGEPKLERAFMDGSNRKDLVKTKLGWPAGVTLDMISKRVYWVDSRFDYIETVTYDGIQRKTVVHGGSLIPHPFGVSLFEGQVFFTDWTKMAVLKANKFTETNPQVYYQASLRPYGVTVYHSLRQPYATNPCKDNNGGCEQVCVLSHRTDNDGLGFRCKCTFGFQLDTDERHCIAVQNFLIFSSQVAIRGIPFTLSTQEDVMVPVSGNPSFFVGIDFDAQDSTIFFSDMSKHMIFKQKIDGTGREILAANRVENVESLAFDWISKNLYWTDSHYKSISVMRLADKTRRTVVQYLNNPRSVVVHPFAGYLFFTDWFRPAKIMRAWSDGSHLLPVINTTLGWPNGLAIDWAASRLYWVDAYFDKIEHSTFDGLDRRRLGHIEQMTHPFGLAIFGEHLFFTDWRLGAIIRVRKADGGEMTVIRSGIAYILHLKSYDVNIQTGSNACNQPTHPNGDCSHFCFPVPNFQRVCGCPYGMRLASNHLTCEGDPTNEPPTEQCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCKVTASESLLLLVASQNKIIADSVTSQVHNIYSLVENGSYIVAVDFDSISGRIFWSDATQGKTWSAFQNGTDRRVVFDSSIILTETIAIDWVGRNLYWTDYALETIEVSKIDGSHRTVLISKNLTNPRGLALDPRMNEHLLFWSDWGHHPRIERASMDGSMRTVIVQDKIFWPCGLTIDYPNRLLYFMDSYLDYMDFCDYNGHHRRQVIASDLIIRHPYALTLFEDSVYWTDRATRRVMRANKWHGGNQSVVMYNIQWPLGIVAVHPSKQPNSVNPCAFSRCSHLCLLSSQGPHFYSCVCPSGWSLSPDLLNCLRDDQPFLITVRQHIIFGISLNPEVKSNDAMVPIAGIQNGLDVEFDDAEQYIYWVENPGEIHRVKTDGTNRTVFASISMVGPSMNLALDWISRNLYSTNPRTQSIEVLTLHGDIRYRKTLIANDGTALGVGFPIGITVDPARGKLYWSDQGTDSGVPAKIASANMDGTSVKTLFTGNLEHLECVTLDIEEQKLYWAVTGRGVIERGNVDGTDRMILVHQLSHPWGIAVHDSFLYYTDEQYEVIERVDKATGANKIVLRDNVPNLRGLQVYHRRNAAESSNGCSNNMNACQQICLPVPGGLFSCACATGFKLNPDNRSCSPYNSFIVVSMLSAIRGFSLELSDHSETMVPVAGQGRNALHVDVDVSSGFIYWCDFSSSVASDNAIRRIKPDGSSLMNIVTHGIGENGVRGIAVDWVAGNLYFTNAFVSETLIEVLRINTTYRRVLLKVTVDMPRHIVVDPKNRYLFWADYGQRPKIERSFLDCTNRTVLVSEGIVTPRGLAVDRSDGYVYWVDDSLDIIARIRINGENSEVIRYGSRYPTPYGITVFENSIIWVDRNLKKIFQASKEPENTEPPTVIRDNINWLRDVTIFDKQVQPRSPAEVNNNPCLENNGGCSHLCFALPGLHTPKCDCAFGTLQSDGKNCAISTENFLIFALSNSLRSLHLDPENHSPPFQTINVERTVMSLDYDSVSDRIYFTQNLASGVGQISYATLSSGIHTPTVIASGIGTADGIAFDWITRRIYYSDYLNQMINSMAEDGSNRTVIARVPKPRAIVLDPCQGYLYWADWDTHAKIERATLGGNFR

SEQ ID NO: 13 - protein, 3623 aa, Homo sapiens
MMNMSLPFLWSLLTLLIFAEVNGEAGELELQRQKRSINLQQPRMATERGNLVFLTGSAQNIEFRTGSLGKIKLNDEDLSECLHQIQKNKEDIIELKGSAIGLPQNISSQIYQLNSKLVDLERKFQGLQQTVDKKVCSSNPCQNGGTCLNLHDSFFCICPPQWKGPLCSADVNECEIYSGTPLSCQNGGTCVNTMGSYSCHCPPETYGPQCASKYDDCEGGSVARCVHGICEDLMREQAGEPKYSCVCDAGWMFSPNSPACTLDRDECSFQPGPCSTLVQCFNTQGSFYCGACPTGWQGNGYICEDINECEINNGGCSVAPPVECVNTPGSSHCQACPPGYQGDGRVCTLTDICSVSNGGCHPDASCSSTLGSLPLCTCLPGYTGNGYGPNGCVQLSNICLSHPCLNGQCIDTVSGYFCKCDSGWTGVNCTENINECLSNPCLNGGTCVDGVDSFSCECTRLWTGALCQVPQQVCGESLSGINGSFSYRSPDVGYVHDVNCFWVIKTEMGKVLRITFTFFRLESMDNCPHEFLQVYDGDSSSAFQLGRFCGSSLPHELLSSDNALYFHLYSEHLRNGRGFTVRWETQQPECGGILTGPYGSIKSPGYPGNYPPGRDCVWIVVTSPDLLVTFTFGTLSLEHHDDCNKDYLEIRDGPLYQDPLLGKFCTTFSVPPLQTTGPFARIHFHSDSQISDQGFHITYLTSPSDLRCGGNYTDPEGELFLPELSGPFTHTRQCVYMMKQPQGEQIQINFTHVELQCQSDSSQNYIEVRDGETLLGKVCGNGTISHIKSITNSVWIRFKIDASVEKASFRAVYQVACGDELTGEGVIRSPFFPNVYPGERTCRWTIHQPQSQVILLNFTVFEIGSSAHCETDYVEIGSSSILGSPENKKYCGTDIPSFITSVYNFLYVTFVKSSSTENHGFMAKFSAEDLACGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFVYGCGGELSGATGSFSSPGFPNRYPPNKECIWYIRTDPGSSIQLTIHDFDVEYHSRCNFDVLEIYGGPDFHSPRIAQLCTQRSPENPMQVSSTGNELAIRFKTDLSINGRGFNASWQAVTGGCGGIFQAPSGEIHSPNYPSPYRSNTDCSWVIRVDRNHRVLLNFTDFDLEPQDSCIMAYDGLSSTMSRLARTCGREQLANPIVSSGNSLFLRFQSGPSRQNRGFRAQFRQACGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNRLQLSFISFQLEDSQDCSRDFVEIREGNATGHLVGRYCGNSFPLNYSSIVGHTLWVRFISDGSGSGTGFQATFMKIFGNDNIVGTHGKVASPFWPENYPHNSNYQWTVNVNASHVVHGRILEMDIEEIQNCYYDKLRIYDGPSIHARLIGAYCGTQTESFSSTGNSLTFHFYSDSSISGKGFLLEWFAVDAPDGVLPTIAPGACGGFLRTGDAPVFLFSPGWPDSYSNRVDCTWLIQAPDSTVELNILSLDIESHRTCAYDSLVIRDGDNNLAQQLAVLCGREIPGPIRSTGEYMFIRFTSDSSVTRAGFNASFHKSCGGYLHADRGIITSPKYPETYPSNLNCSWHVLVQSGLTIAVHFEQPFQIPNGDSSCNQGDYLVLRNGPDICSPPLGPPGGNGHFCGSHASSTLFTSDNQMFVQFISDHSNEGQGFKIKYEAKSLACGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFESSMEECGGDLQGSIGTFTSPNYPNPNPHGRICEWRITAPEGRRITLMFNNLRLATHPSCNNEHVIVFNGIRSNSPQLEKLCSSVNVSNEIKSSGNTMKVIFFTDGSRPYGGFTASYTSSEDAVCGGSLPNTPEGNFTSPGYDGVRNYSRNLNCEWTLSNPNQGNSSISIHFEDFYLESHQDCQFDVLEFRVGDADGPLMWRLCGPSKPTLPLVIPYSQVWIHFVTNERVEHIGFHAKYSFTDCGGIQIGDSGVITSPNYPNAYDSLTHCSSLLEAPQGHTITLTFSDFDIEPHTTCAWDSVTVRNGGSPESPIIGQYCGNSNPRTIQSGSNQLVVTFNSDHSLQGGGFYATWNTQTLGCGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRIISCGGVFNFSSGIITSPAYSYADYPNDMHCLYTITVSDDKVIELKFSDFDVVPSTSCSHDYLAIYDGANTSDPLLGKFCGSKRPPNVKSSNNSMLLVFKTDSFQTAKGWKMSFRQTLGPQQGCGGYLTGSNNTFASPDSDSNGMYDKNLNCVWIIIAPVNKVIHLTFNTFALEAASTRQRCLYDYVKLYDGDSENANLAGTFCGSTVPAPFISSGNFLTVQFISDLTLEREGFNATYTIMDMPCGGTYNATWTPQNISSPNSSDPDVPFSICTWVIDSPPHQQVKITVWALQLTSQDCTQNYLQLQDSPQGHGNSRFQFCGRNASAVPVFYSSMSTAMVIFKSGVVNRNSRMSFTYQIADCNRDYHKAFGNLRSPGWPDNYDNDKDCTVTLTAPQNHTISLFFHSLGIENSVECRNDFLEVRNGSNSNSPLLGKYCGTLLPNPVFSQNNELYLRFKSDSVTSDRGYEIIWTSSPSGCGGTLYGDRGSFTSPGYPGTYPNNTYCEWVLVAPAGRLVTINFYFISIDDPGDCVQNYLTLYDGPNASSPSSGPYCGGDTSIAPFVASSNQVFIKFHADYARRPSAFRLTWDS

SEQ ID NO: 14 - protein, 332 aa, Homo sapiens
SSNPCQNGGTCLNLHDSFFCICPPQWKGPLCSADVNECEIYSGTPLSCQNGGTCVNTMGSYSCHCPPETYGPQCASKYDDCEGGSVARCVHGICEDLMREQAGEPKYSCVCDAGWMFSPNSPACTLDRDECSFQPGPCSTLVQCFNTQGSFYCGACPTGWQGNGYICEDINECEINNGGCSVAPPVECVNTPGSSHCQACPPGYQGDGRVCTLTDICSVSNGGCHPDASCSSTLGSLPLCTCLPGYTGNGYGPNGCVQLSNICLSHPCLNGQCIDTVSGYFCKCDSGWTGVNCTENINECLSNPCLNGGTCVDGVDSFSCECTRLWTGALCQ

SEQ ID NO: 15 - protein, 802 aa, Homo sapiens
CGESLSGINGSFSYRSPDVGYVHDVNCFWVIKTEMGKVLRITFTFFRLESMDNCPHEFLQVYDGDSSSAFQLGRFCGSSLPHELLSSDNALYFHLYSEHLRNGRGFTVRWETQQPECGGILTGPYGSIKSPGYPGNYPPGRDCVWIVVTSPDLLVTFTFGTLSLEHHDDCNKDYLEIRDGPLYQDPLLGKFCTTFSVPPLQTTGPFARIHFHSDSQISDQGFHITYLTSPSDLRCGGNYTDPEGELFLPELSGPFTHTRQCVYMMKQPQGEQIQINFTHVELQCQSDSSQNYIEVRDGETLLGKVCGNGTISHIKSITNSVWIRFKIDASVEKASFRAVYQVACGDELTGEGVIRSPFFPNVYPGERTCRWTIHQPQSQVILLNFTVFEIGSSAHCETDYVEIGSSSILGSPENKKYCGTDIPSFITSVYNFLYVTFVKSSSTENHGFMAKFSAEDLACGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYR

SEQ ID NO: 16 - protein, 457 aa, Homo sapiens
CGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFV

SEQ ID NO: 17 - protein, 791 aa, Homo sapiens
GTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFVYGCGGELSGATGSFSSPGFPNRYPPNKECIWYIRTDPGSSIQLTIHDFDVEYHSRCNFDVLEIYGGPDFHSPRIAQLCTQRSPENPMQVSSTGNELAIRFKTDLSINGRGFNASWQAVTGGCGGIFQAPSGEIHSPNYPSPYRSNTDCSWVIRVDRNHRVLLNFTDFDLEPQDSCIMAYDGLSSTMSRLARTCGREQLANPIVSSGNSLFLRFQSGPSRQNRGFRAQFRQACGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNRLQLSFISFQLEDSQDCSRDFVEIREGNATGHLVGRYCGNSFPLNYSSIVGHTLWVRFISDGSGSGTGFQATF

SEQ ID NO: 18 - protein, 828 aa, Homo sapiens
CGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNRLQLSFISFQLEDSQDCSRDFVEIREGNATGHLVGRYCGNSFPLNYSSIVGHTLWVRFISDGSGSGTGFQATFMKIFGNDNIVGTHGKVASPFWPENYPHNSNYQWTVNVNASHVVHGRILEMDIEEIQNCYYDKLRIYDGPSIHARLIGAYCGTQTESFSSTGNSLTFHFYSDSSISGKGFLLEWFAVDAPDGVLPTIAPGACGGFLRTGDAPVFLFSPGWPDSYSNRVDCTWLIQAPDSTVELNILSLDIESHRTCAYDSLVIRDGDNNLAQQLAVLCGREIPGPIRSTGEYMFIRFTSDSSVTRAGFNASFHKSCGGYLHADRGIITSPKYPETYPSNLNCSWHVLVQSGLTIAVHFEQPFQIPNGDSSCNQGDYLVLRNGPDICSPPLGPPGGNGHFCGSHASSTLFTSDNQMFVQFISDHSNEGQGFKIKYEAKSLACGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFES

SEQ ID NO: 19 - protein, 818 aa, Homo sapiens
CGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFESSMEECGGDLQGSIGTFTSPNYPNPNPHGRICEWRITAPEGRRITLMFNNLRLATHPSCNNEHVIVFNGIRSNSPQLEKLCSSVNVSNEIKSSGNTMKVIFFTDGSRPYGGFTASYTSSEDAVCGGSLPNTPEGNFTSPGYDGVRNYSRNLNCEWTLSNPNQGNSSISIHFEDFYLESHQDCQFDVLEFRVGDADGPLMWRLCGPSKPTLPLVIPYSQVWIHFVTNERVEHIGFHAKYSFTDCGGIQIGDSGVITSPNYPNAYDSLTHCSSLLEAPQGHTITLTFSDFDIEPHTTCAWDSVTVRNGGSPESPIIGQYCGNSNPRTIQSGSNQLVVTFNSDHSLQGGGFYATWNTQTLGCGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRI

SEQ ID NO: 20 - protein, 817 aa, Homo sapiens
CGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRIISCGGVFNFSSGIITSPAYSYADYPNDMHCLYTITVSDDKVIELKFSDFDVVPSTSCSHDYLAIYDGANTSDPLLGKFCGSKRPPNVKSSNNSMLLVFKTDSFQTAKGWKMSFRQTLGPQQGCGGYLTGSNNTFASPDSDSNGMYDKNLNCVWIIIAPVNKVIHLTFNTFALEAASTRQRCLYDYVKLYDGDSENANLAGTFCGSTVPAPFISSGNFLTVQFISDLTLEREGFNATYTIMDMPCGGTYNATWTPQNISSPNSSDPDVPFSICTWVIDSPPHQQVKITVWALQLTSQDCTQNYLQLQDSPQGHGNSRFQFCGRNASAVPVFYSSMSTAMVIFKSGVVNRNSRMSFTYQIADCNRDYHKAFGNLRSPGWPDNYDNDKDCTVTLTAPQNHTISLFFHSLGIENSVECRNDFLEVRNGSNSNSPLLGKYCGTLLPNPVFSQNNELYLRFKSDSVTSDRGYEIIWTSSPSGCGGTLYGDRGSFTSPGYPGTYPNNTYCEWVLVAPAGRLVTINFYFISIDDPGDCVQNYLTLYDGPNASSPSSGPYCGGDTSIAPFVASSNQVFIKFHADYARRPSAFRLTW